(19) United States
(12) Patent Application Publication
(10) Pub. No.: US 2010/0210655 A1
(43) Pub. Date: Aug. 19, 2010

(54) NOVEL sEH INHIBITORS AND THEIR USE

(76) Inventors: Yun Ding, Waltham, MA (US); Allyn T. Londregan, Barrington, RI (US); Joseph Paul Marino, JR., King of Prussia, PA (US)

Correspondence Address: GlaxoSmithKline;GLOBAL PATENTS -US, UW2220, P. O. BOX 1539, KING OF PRUSSIA, PA, 19406-0939, US

(21) Appl. No.: 12/682,068

(22) PCT Filed: Oct. 10, 2008
(86) PCT No.: PCT/US08/79514

§ 371 Date: Apr. 8, 2010

Related U.S. Application Data
(60) Provisional application No. 60/979,139, filed on Oct. 11, 2007.

(51) Int. Cl. A61K 31/53; C07D 403/14; C07D 403/04; A61P 9/00; A61P 1/16; A61P 11/06; A61P 3/10; A61P 3/04

(52) U.S. Cl. 514/245; 544/198; 544/209

(57) ABSTRACT

The invention is directed to novel sEH inhibitors and their use in the treatment of diseases mediated by the sEH enzyme. Specifically, the invention is directed to compounds according to Formula I:

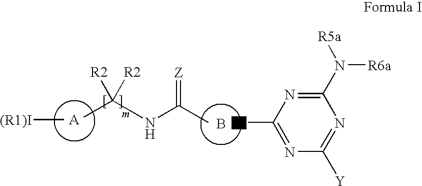

wherein R1, R2, R5a, R6a, A, B, Y, I, and m are defined below, and to pharmaceutically-acceptable salts thereof. The compounds of the invention are sEH inhibitors and can be used in the treatment of diseases mediated by the sEH enzyme, such as hypertension. Accordingly, the invention is further directed to pharmaceutical compositions comprising a compound of the invention. The invention is still further directed to methods of inhibiting sEH and treatment of conditions associated therewith using a compound of the invention or a pharmaceutical composition comprising a compound of the invention.

### FIELD OF THE INVENTION

The invention is directed to novel sEH inhibitors and their use in the treatment of diseases mediated by the sEH enzyme.

### BACKGROUND OF THE INVENTION

Epoxide functional groups may be found in drugs, xenobiotic materials, and endogenous biomolecules. Epoxide hydrolases, found in both plants and animals, are enzymes that convert epoxides to diols by hydrolysis. In mammals, soluble epoxide hydrolase (“sEH”) is primarily responsible for the metabolism of arachidonic acid derivatives known as epoxyeicosatrienoic acids (“EETs”). sEH converts EETs into dihydroxyeicosatrienoic acids (“DHETs”). Several publications have described the beneficial vasodilatory, anti-inflamatory, and anti-thrombotic effects of EETs. See E.g. Spector et al., Prog. Lipid Res., 43, 55-90, 2004; Imig, Cardiovasc. Drug Rev., 24, 169-188, 2006. DHETs are generally inactive and thus do not exhibit the beneficial effects of EETs.

Conversely, microsomal epoxide hydrolase (“mEH”) catalyzes the hydrolysis of a broad range of epoxide substrates including carcinogenic polycyclic aromatic hydrocarbons and reactive epoxides, thus it provides an important detoxification pathway. Polymorphisms in mEH may lead to differences in bioactivation of pro-carcinogens and several human epidemiological studies suggest that mEH genotype is associated with altered cancer risk. Fretland & Omiecinski, Chemico-Biol. Int., 129, 41-59, 2000.

Pharmacological, knockout mouse phenotype and genetic polymorphism studies suggest that elevated EET levels are protective in numerous cardiovascular disorders including hypertension [Sinal et al., J. Biol. Chem., 275, 40504-40510, 2000; Imig et al., Hypertension, 39, 690-694, 2002; Jung et al., Hypertension, 45, 759-765, 2005; Loch et al., Cell Biochem Biophys., 47, 87-98, 2007], heart failure [Xu et al., Proc. Natl. Acad. Sci. U.S.A. 103, 18733-18738, 2006], renal dysfunction/end organ damage [Zhao et al. J. Am. Soc. Nephrol., 15; 1244-1253, 2004; Imig et al., Hypertension, 46; 975-981, 2005], stroke [Dorrance et al., J. Cardiovasc. Pharmacol., 46; 842-848, 2005; Formage et al., Hum. Mol. Genet., 14; 2829-2837, 2005; Koerner et al., J. Neurosci., 27; 4642-4649, 2007], atherosclerosis and thrombosis [Sato et al., J. Hum. Genet., 49; 29-34, 2004; Lee et al., Hum Mol Genet., 15; 1640-1649, 2006; Wei et al., Atherosclerosis, 190; 26-34, 2007; Krotz et al., Arterioscler. Thromb. Vasc. Biol., 24; 595-600, 2004] and inflammation [Inceoglu et al., Life Sci., 79; 2311-2319, 2006].

One approach to the treatment of such conditions designed to take advantage of the beneficial effect of EETs has been to inhibit the action of sEH thereby preventing EET degradation. In light of the role sEH plays in the degradation of EETs, it is desirable to prepare compounds that inhibit its activity. Thus, there is a need to identify compounds that inhibit sEH, which can be used in the treatment of a variety of conditions mediated by the sEH enzyme.

### SUMMARY OF THE INVENTION

The invention is directed to novel sEH inhibitors and their use in the treatment of diseases mediated by the sEH enzyme. Specifically, the invention is directed to compounds according to Formula I:

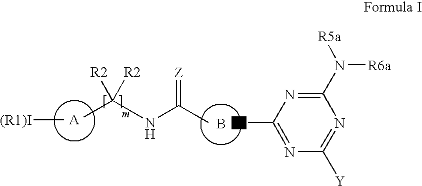

wherein R1, R2, R5a, R6a, A, B, Y, I, and m are defined below, and to pharmaceutically-acceptable salts thereof.

The compounds of the invention are sEH inhibitors and can be used in the treatment of diseases mediated by the sEH enzyme, such as hypertension. Accordingly, the invention is further directed to pharmaceutical compositions comprising a compound of the invention. The invention is still further directed to methods of inhibiting sEH and treatment of conditions associated therewith using a compound of the invention or a pharmaceutical composition comprising a compound of the invention.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical and biological arts. For example, the following abbreviations are used herein:

“aq” is an abbreviation for aqueous

“BOP” is an abbreviation for (Benzotriazol-1-yloxy)tris (dimethylamino)phosphonium hexafluorophosphate

“° C.” is an abbreviation for degrees Celsius

“DIEA” is an abbreviation for di-isopropylethylamine

“DMAP” is an abbreviation for dimethylaminopyridine

“DMF” is an abbreviation for dimethylformamide

“DMSO” is an abbreviation for Dimethylsulfoxide

“EDCl” is an abbreviation for N-(3-Dimethylaminopropyl)-N′-ethylcarbodiimide hydrochloride

“eqiv” is an abbreviation for equivalent

“HPLC” is an abbreviation for High Pressure Liquid Chromatography

“g” is an abbreviation for gram or grams

“L” is an abbreviation for liter or liters

“LC-MS” is an abbreviation for Liquid chromatography-Mass spectrometry

“mL” is an abbreviation for milliliter or milliliters

“min” is an abbreviation for minute or minutes

“mmol” is an abbreviation for millimole or millimolar

“N” is an abbreviation for Normal and refers to the number of equivalents of reagent per liter of solution

“Ph” is an abbreviation for phenyl

“sat” is an abbreviation for saturated

“TFA” is an abbreviation for trifluoroacetic acid

“THF” is an abbreviation for tetrahydrofuran

### TERMS AND DEFINITIONS

“Alkyl” refers to a monovalent saturated hydrocarbon chain having the specified number of member atoms. For example, C1-C8 alkyl refers to an alkyl group having from 1 to 8 member atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Cycloalkyl” refers to a monovalent saturated or unsaturated hydrocarbon ring having the specified number of member atoms. For example, C3-C6 cycloalkyl refers to a cycloalkyl group having from 3 to 6 member atoms. Unsaturated Cycloalkyl groups have one or more carbon-carbon double bonds within the ring. Cycloalkyl groups are not aromatic. Cycloalkyl groups having from 3 to 7 member atoms or less are monocyclic ring systems. Cycloalkyl groups having at least 7 member atoms may be monocyclic, bridged or fused bicyclic ring systems. Cycloalkyl groups may be optionally substituted with one or more substituents as defined herein. Cycloalkyl includes cyclopropyl, cyclopropenyl, cyclobutyl, cyclobutenyl, cyclopentyl, cyclopentenyl, cyclohexyl, cyclohexenyl, cycloheptanyl, and cycloheptenyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” refers to the time required for half of a quantity of a substance to be converted to another chemically distinct specie in vitro or in vivo.

“Halo” refers to the halogen radical fluoro, chloro, bromo, or iodo.

“Haloalkyl” refers to an alkyl group that is substituted with one or more halo substituents. Haloalkyl includes trifluoromethyl.

“Heteroaryl” refers to a monovalent aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Unless otherwise specified, heteroaryl groups are monocyclic ring systems or are fused, spiro, or bridged bicyclic ring systems. Monocyclic heteroaryl rings have 5 or 6 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, oxadiazolyl, thiazolyl, isothiazolyl, thiadiazolyl, furanyl, furazanyl, thienyl, triazolyl, pyridinyl, pyrimidinyl, pyridazinyl, pyrazinyl, triazinyl, tetrazinyl, tetrazolyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benzopyranyl, benzoxazolyl, benzisoxazolyl, benzofuranyl, isobenzofuranyl, benzothiazolyl, benzisothiazolyl, benzothienyl, furopyridinyl, and napthyridinyl.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituent as defined herein. Unless otherwise specified, heterocycloalkyl groups are monocyclic, bridged, or fused ring systems. Monocyclic heterocycloalkyl rings have from 4 to 7 member atoms. Bridged or bicyclic heterocycloalkyl rings have from 7 to 11 member atoms. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azetidinyl, azabicyclo[3.2.1]octyl, azabicyclo[3.3.1]nonyl, azabicyclo[4.3.0]nonyl, oxabicylo[2.2.1]heptyl, and phthalimidyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

### Compounds

The invention is directed to compounds according to Formula I:

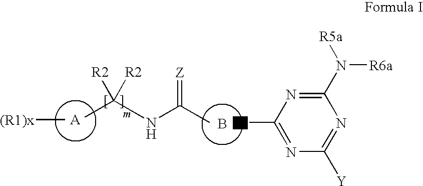

wherein:

A is phenyl, monocyclic heteroaryl, or C5-C6 cycloalkyl;

when A is phenyl or monocyclic heteroaryl each R1 is independently selected from the group consisting of: halo, CN, Ra, ORb, C(O)ORc, C(O)NRcRc, NRcRc, NRcC(O)Rb, NRcS(O₂)Ra, SRb, S(O₂)Ra, and S(O₂)NRcRc;

when A is C5-C6 cycloalkyl each R1 is independently selected from the group consisting of: Ra, ORb, C(O)ORc, C(O)NRcRc, NRcRc, and NRcC(O)Rb;

x is an integer from 0 to 5;

each R2 is independently H or C1-C3 alkyl;

m is 1 or 2;

Z is O or S;

B is B1, B2, B3, B4, B5, B6, B7, B8, B9, B10, B11, or B12;

B1 is

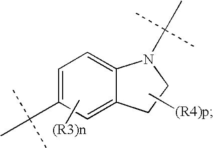

B2 is

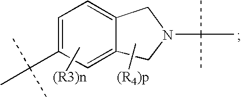

B3 is

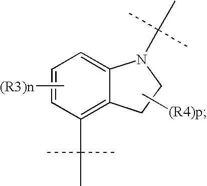

B4 is

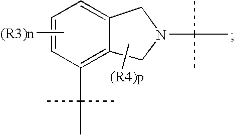

B5 is

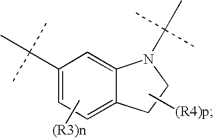

B6 is

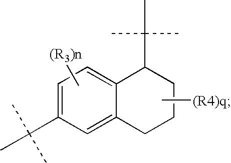

B7 is

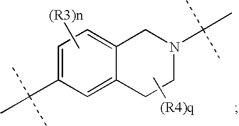

B8 is

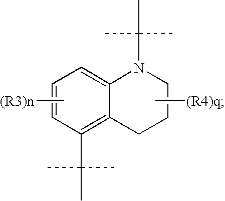

B9 is

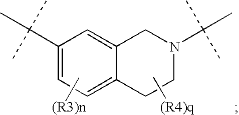

B10 is

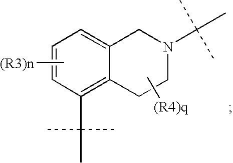

B11 is

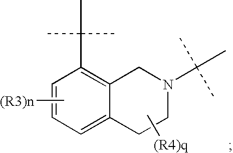

B12 is

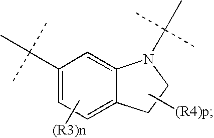

R3, if present, is a substituent on the phenyl ring of said B ring system and each R3 is independently selected from the group consisting of: halo and C1-C3 alkyl;

n is an integer from 0 to 3;

R4, if present, is a substituent on the Nitrogen-containing ring of said B ring system and each R4 is independently C1-C3 alkyl;

p is an integer from 0 to 2;

q is an integer from 0 to 4;

Y is H, OH, R7, R8, R9, R10, R11, R12, or NR5bR6b;

R5a and R5b are each independently H, R51, R52, R53, R54, R55, —C(O)Rb, —C(O)NRcRc, —S(O₂)Ra, or —S(O₂)NRcRc;

each R51 is C1-C6 alkyl optionally substituted with one or more substituents selected from the group consisting of: halo, ORd, SRk, C(O)ORc, C(O)NReRe, NReRe, Rg, Rh, Ri, Rj;

each R52 is C3-C6 cycloalkyl optionally substituted with one or more substituents selected from the group consisting of: halo, ORd, SRd, C(O)ORc, C(O)NReRe, NReRe, C1-C3 alkyl, and C1-C3 haloalkyl;

R53 is monocyclic heterocycloalkyl optionally substituted with one or more C1-C3 alkyl;

R54 is phenyl optionally substituted with one or more substituents selected from the group consisting of: halo, CN, Ra, ORb, C(O)ORc, C(O)NRcRc, NRcRc, NRcC(O)Rb, NRcS(O₂)Ra, SRb, S(O₂)Ra, and S(O₂)NReRe;

R55 is monocyclic heteroaryl optionally substituted with one or more substituents selected from the group consisting of: halo, —CN, C1-C3 alkyl, C1-C3 haloalkyl, ORd, and NReRe;

R6a and R6b are each independently H, R51, or R52; or

R5a and R6a and/or R5b and R6b, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;

R7 is C1-C8 alkyl optionally substituted with one or more substituents selected from the group consisting of: halo, ORd, SRd, NReRe, C3-C6 cycloalkyl, Ri, and Rj;

R8 is C3-C6 cycloalkyl optionally substituted with one or more substituents selected from the group consisting of: halo, ORd, SRd, NReRe, C1-C3 alkyl, and C1-C3 haloalkyl;

R9 monocyclic heterocycloalkyl optionally substituted with one or more C1-C3 alkyl;

R10 is phenyl optionally substituted with one or more substituents selected from the group consisting of: halo, CN, Ra, ORb, C(O)ORc, C(O)NReRe, NReRe, NRcC(O)Rb, NRcS(O₂)Ra, SRb, S(O₂)Ra, and S(O₂)NRcRc

R11 is heteroaryl optionally substituted with one or more substituents selected from the group consisting of: halo, CN, Ra, ORb, C(O)ORc, C(O)NReRe, NReRe, NRcC(O)Rb, NRcS(O₂)Ra, SRb, S(O₂)Ra, and S(O₂)NRcRc;

R12 is —OR7, —OR8, —OR9, —OR10, —OR11, —SR7, —SR8, —SR9, —SR10, or SR11;

each Ra is independently C1-C6 alkyl or C1-C6 haloalkyl;

each Rb is independently H, C1-C6 alkyl or C1-C6 haloalkyl;

each Rc is independently H or C1-C6 alkyl;

where there are two Rc groups attached to a nitrogen;

both Rc groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;

each Rd is independently H, C1-C3 alkyl or C1-C3 haloalkyl;

each Re is independently H, C1-C3 alkyl, CH₂—CF₃; or

both Re groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;

each Rf is independently H or C1-C3 alkyl.

each Rg is C3-C6 cycloalkyl optionally substituted with one or more substituents selected from the group consisting of: halo, ORd, SRd, C(O)ORc, C(O)NReRe, NReRe, and C1-C3 alkyl;

each Rh is monocyclic heterocycloalkyl optionally substituted with one or more C1-C3 alkyl;

each Ri is phenyl optionally substituted with one or more substituents selected from the group consisting of: halo, —CN, C1-C3 alkyl, C1-C3 haloalkyl, ORd, and NReRe;

each Rj is monocyclic heteroaryl optionally substituted with one or more substituents selected from the group consisting of: halo, —CN, C1-C3 alkyl, C1-C3 haloalkyl, ORd, and NReRe; and

each Rk is independently H, C1-C3 alkyl, C1-C3 haloalkyl, or benzyl optionally substituted with one or more substituents selected from the group consisting of: halo, —CN, C1-C3 alkyl, C1-C3 haloalkyl, ORd, and NReRe.

The meaning of any functional group or substituent thereon at any one occurrence in Formula I, or any subformula thereof, is independent of its meaning, or any other functional group's or substituent's meaning, at any other occurrence, unless stated otherwise.

The compounds according to Formula I may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass any stereoisomer and all mixtures thereof. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. Likewise, all tautomeric forms are also included in Formula I whether such tautomers exist in equilibrium or predominately in one form.

In certain embodiments, compounds according to Formula I may contain an acidic functional group and are therefore capable of forming pharmaceutically-acceptable base addition salts by treatment with a suitable base. In certain other embodiments, compounds according to Formula I may contain a basic functional group and are therefore capable of forming pharmaceutically-acceptable acid addition salts by treatment with a suitable acid. Thus, the skilled artisan will appreciate that pharmaceutically-acceptable salts of the compounds according to Formula I may be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

Because of their potential use in medicine, the salts of the compounds of formula (I) are preferably pharmaceutically acceptable. Suitable pharmaceutically acceptable salts can include acid or base addition salts.

A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt can be formed by reaction of a compound of formula (I) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I).

Other non-pharmaceutically acceptable salts, eg. oxalates, may be used, for example in the isolation of compounds of the invention, and are included within the scope of this invention.

The invention includes within its scope all possible stoichiometric and non-stoichiometric forms of the salts of the compounds of formula (I).

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

In the solid state, compounds of the invention can exist in crystalline, semi-crystalline and amorphous forms, as well as mixtures thereof. The skilled artisan will appreciate that pharmaceutically-acceptable solvates of a compound of the invention may be formed wherein solvent molecules are incorporated into the solid-state structure during crystallization. Solvates may involve water or nonaqueous solvents, or mixtures thereof. In addition, the solvent content of such solvates can vary in response to environment and upon storage. For example, water may displace another solvent over time depending on relative humidity and temperature.

Solvates wherein water is the solvent that is incorporated into the solid-state structure are typically referred to as “hydrates.” Solvates wherein more than one solvent is incorporated into the solid-state structure are typically referred to as “mixed solvates”. Solvates include “stoichiometric solvates” as well as compositions containing variable amounts of solvent (referred to as “non-stoichiometric solvates”). Stoichiometric solvates wherein water is the solvent that is incorporated into the solid-state structure are typically referred to as “stoichiometric hydrates”, and non-stoichiometric solvates wherein water is the solvent that is incorporated into the solid-state structure are typically referred to as “non-stoichiometric hydrates”. The invention includes both stoichiometric and non-stoichiometric solvates.

In addition, crystalline forms of a compound of the invention, including solvates thereof, may contain solvent molecules, which are not incorporated into the solid-state structure. For example, solvent molecules may become trapped in the crystals upon isolation. In addition, solvent molecules may be retained on the surface of the crystals. The invention includes such forms.

The skilled artisan will further appreciate that compounds of the invention, including solvates thereof, may exhibit polymorphism (i.e. the capacity to occur in different crystalline packing arrangements). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different IR spectra and X-ray powder diffraction patterns, which may be used for identification. Polymorphs may also exhibit different melting points, which may be used for identification. The skilled artisan will appreciate that different polymorphs may be produced, for example, by changing or adjusting the reaction conditions or reagents, used in making the compound. For example, changes in temperature, pressure, or solvent may result in the production of different polymorphs. In addition, one polymorph may spontaneously convert to another polymorph under certain conditions.

### Representative Embodiments

In one embodiment:

A is phenyl, thiophenyl, or pyridyl;

R1 is CF₃, halo, OCF₃, CN, OC₁-C₆ alkyl, morpholino, CO₂H, or N(CH₃)₂;

x is 1, 2, or 3;

B is B1, B2, B6, and B7;

n is 0;

Z is O;

Y is C1-C3 alkyl, phenyl, thiophenyl, or pyridyl; wherein the phenyl, thiophenyl or pyridyl may be substituted by —CO₂H, SO₂Me, CF₃, halo, or CN;

R5a is hydrogen or C1-C6 alkyl; and

R6a is hydrogen or C1-C6 alkyl;

or a pharmaceutically acceptable salt thereof.

In another embodiment:

A is phenyl;

R1 is CF₃, halo, OCF₃, CN, OC₁-C₆ alkyl, or morpholino;

x is 1, or 2;

B is;

n is 0

Z is O;

Y is methyl;

R5a is hydrogen; and

R6a is;

or a pharmaceutically acceptable salt thereof.

Specific examples of compounds of the present invention include the following:
N-[(2,4-dichlorophenyl)methyl]-2-[4-(methylamino)-6-(4-methyl-1-piperazinyl)-1,3,5-triazin-2-yl]-2,3-dihydro-1H-isoindole-5-carboxamide;
2-[4-(methylamino)-6-(4-methyl-1-piperazinyl)-1,3,5-triazin-2-yl]-N-{[2-(trifluoromethyl)phenyl]methyl}-2,3-dihydro-1H-isoindole-5-carboxamide trifluoroacetate;
2-[4-(methylamino)-6-(4-methyl-1-piperazinyl)-1,3,5-triazin-2-yl]-N-({2-[(trifluoromethyl)oxy]phenyl}methyl)-2,3-dihydro-1H-isoindole-5-carboxamide trifluoroacetate;
N-{[4-chloro-2-(trifluoromethyl)phenyl]methyl}-2-[4-(methylamino)-6-(4-methyl-1-piperazinyl)-1,3,5-triazin-2-yl]-2,3-dihydro-1H-isoindole-5-carboxamide trifluoroacetate;
2-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-isoquinolinecarboxamide;
2-[4-(methylamino)-6-(4-methyl-1-piperazinyl)-1,3,5-triazin-2-yl]-N-{[2-(trifluoromethyl)phenyl]methyl}-1,2,3,4-tetrahydro-6-isoquinolinecarboxamide;
2-[4-(methylamino)-6-(4-methyl-1-piperazinyl)-1,3,5-triazin-2-yl]-N-{[4-(trifluoromethyl)phenyl]methyl}-1,2,3,4-tetrahydro-6-isoquinolinecarboxamide;
N-[(2,5-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide;
4-{[({2-[4-(methylamino)-6-(4-methyl-1-piperazinyl)-1,3,5-triazin-2-yl]-1,2,3,4-tetrahydro-6-isoquinolinyl}carbonyl)amino]methyl}benzoic acid;
N-[(2,4-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide;
N-{[4-chloro-2-(trifluoromethyl)phenyl]ethyl}-2-[4-(methylamino)-6-(4-methyl-1-piperazinyl)-1,3,5-triazin-2-yl]-1,2,3,4-tetrahydro-6-isoquinolinecarboxamide;
1-[4-(methylamino)-6-(4-methyl-1-piperazinyl)-1,3,5-triazin-2-yl]-N-{[2-(trifluoromethyl)phenyl]ethyl}-1,2,3,4-tetrahydro-6-quinolinecarboxamide;
1-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-quinolinecarboxamide;
N-[(2,4-dichlorophenyl)methyl]-1-[4-(methylamino)-6-(4-methyl-1-piperazinyl)-1,3,5-triazin-2-yl]-1,2,3,4-tetrahydro-6-quinolinecarboxamide;
N-[(2,5-dichlorophenyl)methyl]-1-[4-(methylamino)-6-(4-methyl-1-piperazinyl)-1,3,5-triazin-2-yl]-1,2,3,4-tetrahydro-6-quinolinecarboxamide;
1-[4-(methylamino)-6-(4-methyl-1-piperazinyl)-1,3,5-triazin-2-yl]-N-{[4-(trifluoromethyl)phenyl]methyl}-1,2,3,4-tetrahydro-6-quinolinecarboxamide;
1-[4-(methylamino)-6-(4-methyl-1-piperazinyl)-1,3,5-triazin-2-yl]-N-{[2-(methyloxy)phenyl]methyl}-1,2,3,4-tetrahydro-6-quinolinecarboxamide;
N-[(3,4-difluorophenyl)methyl]-1-[4-(methylamino)-6-(4-methyl-1-piperazinyl)-1,3,5-triazin-2-yl]-1,2,3,4-tetrahydro-6-quinolinecarboxamide;
1-[4-(methylamino)-6-(4-methyl-1-piperazinyl)-1,3,5-triazin-2-yl]-N-{[2-(trifluoromethyl)phenyl]methyl}-2,3-dihydro-1H-indole-5-carboxamide;
1-[4-(methylamino)-6-(4-methyl-1-piperazinyl)-1,3,5-triazin-2-yl]-N-({2-[(trifluoromethyl)oxy]phenyl}methyl)-2,3-dihydro-1H-indole-5-carboxamide trifluoroacetate;
N-[(2,4-dichlorophenyl)methyl]-1-[4-(methylamino)-6-(4-methyl-1-piperazinyl)-1,3,5-triazin-2-yl]-2,3-dihydro-1H-indole-5-carboxamide trifluoroacetate;
N-{[4-chloro-2-(trifluoromethyl)phenyl]methyl}-1-[4-(methylamino)-6-(4-methyl-1-piperazinyl)-1,3,5-triazin-2-yl]-2,3-dihydro-1H-indole-5-carboxamide trifluoroacetate;
N-({2-[(trifluoromethyl)oxy]phenyl}methyl)-1,2,3,4-tetrahydro-6-isoquinolinecarboxamide 2,4-dichloro-6-phenyl-1,3,5-triazine;
2-[4-(methylamino)-6-phenyl-1,3,5-triazin-2-yl]-N-({2-[(trifluoromethyl)oxy]phenyl}methyl)-1,2,3,4-tetrahydro-6-isoquinolinecarboxamide;
1-[4-(methylamino)-6-phenyl-1,3,5-triazin-2-yl]-N-{[2-(trifluoromethyl)phenyl]methyl}-1,2,3,4-tetrahydro-6-quinolinecarboxamide;
1-[4-(methylamino)-6-phenyl-1,3,5-triazin-2-yl]-N-{[2-(trifluoromethyl)phenyl]methyl}-2,3-dihydro-1H-indole-5-carboxamide;
2-[4-(methylamino)-6-phenyl-1,3,5-triazin-2-yl]-N-{[2-(trifluoromethyl)phenyl]methyl}-1,2,3,4-tetrahydro-6-isoquinolinecarboxamide;
N-[(2,4-dichlorophenyl)methyl]-1-[4-(methylamino)-6-phenyl-1,3,5-triazin-2-yl]-2,3-dihydro-1H-indole-5-carboxamide;
N-[(2,4-dichlorophenyl)methyl]-1-[4-(methylamino)-6-phenyl-1,3,5-triazin-2-yl]-1,2,3,4-tetrahydro-6-quinolinecarboxamide;
1-[4-(methylamino)-6-phenyl-1,3,5-triazin-2-yl]-N-({2-[(trifluoromethyl)oxy]phenyl}methyl)-2,3-dihydro-1H-indole-5-carboxamide;
1-[4-(methylamino)-6-phenyl-1,3,5-triazin-2-yl]-N-({2-[(trifluoromethyl)oxy]phenyl}methyl)-1,2,3,4-tetrahydro-6-quinolinecarboxamide;
N-[(2,4-dichlorophenyl)methyl]-2-[4-(methylamino)-6-phenyl-1,3,5-triazin-2-yl]-2,3-dihydro-1H-isoindole-5-carboxamide;
2-[4-(methylamino)-6-phenyl-1,3,5-triazin-2-yl]-N-{[2-(trifluoromethyl)phenyl]methyl}-2,3-dihydro-1H-isoindole-5-carboxamide;
2-[4-(methylamino)-6-phenyl-1,3,5-triazin-2-yl]-N-({2-[(trifluoromethyl)oxy]phenyl}methyl)-2,3-dihydro-1H-isoindole-5-carboxamide;
N-[(2,4-dichlorophenyl)methyl]-2-[4-(methylamino)-6-phenyl-1,3,5-triazin-2-yl]-1,2,3,4-tetrahydro-6-isoquinolinecarboxamide trifluoroacetate;
1-[4-methyl-6-(methylamino)-1,3,5-triazin-2-yl]-N-({2-[(trifluoromethyl)oxy]phenyl}methyl)-2,3-dihydro-1H-indole-5-carboxamide;
2-[4-methyl-6-(methylamino)-1,3,5-triazin-2-yl]-N-({[2-(trifluoromethyl)oxy]phenyl}methyl)-1,2,3,4-tetrahydro-6-isoquinolinecarboxamide;
1-[4-methyl-6-(methylamino)-1,3,5-triazin-2-yl]-N-({[2-(trifluoromethyl)oxy]phenyl}methyl)-1,2,3,4-tetrahydro-6-quinolinecarboxamide;
2-[4-methyl-6-(methylamino)-1,3,5-triazin-2-yl]-N-{[2-(trifluoromethyl)phenyl]methyl}-1,2,3,4-tetrahydro-6-isoquinolinecarboxamide;
1-[4-methyl-6-(methylamino)-1,3,5-triazin-2-yl]-N-{[2-(trifluoromethyl)phenyl]methyl}-1,2,3,4-tetrahydro-6-quinolinecarboxamide;
1-[4-methyl-6-(methylamino)-1,3,5-triazin-2-yl]-N-{[2-(trifluoromethyl)phenyl]methyl}-2,3-dihydro-1H-indole-5-carboxamide;
N-[(2,4-dichlorophenyl)methyl]-2-[4-methyl-6-(methylamino)-1,3,5-triazin-2-yl]-1,2,3,4-tetrahydro-6-isoquinolinecarboxamide;
N-[(2,4-dichlorophenyl)methyl]-1-[4-methyl-6-(methylamino)-1,3,5-triazin-2-yl]-1,2,3,4-tetrahydro-6-quinolinecarboxamide;
N-[(2,4-dichlorophenyl)methyl]-1-[4-methyl-6-(methylamino)-1,3,5-triazin-2-yl]-2,3-dihydro-1H-indole-5-carboxamide;
2-[4-methyl-6-(methylamino)-1,3,5-triazin-2-yl]-N-{[2-(trifluoromethyl)phenyl]methyl}-2,3-dihydro-1H-isoindole-5-carboxamide;
2-[4-methyl-6-(methylamino)-1,3,5-triazin-2-yl]-N-({2-[(trifluoromethyl)oxy]phenyl}methyl)-2,3-dihydro-1H-isoindole-5-carboxamide;
N-[(2,4-dichlorophenyl)methyl]-2-[4-methyl-6-(methylamino)-1,3,5-triazin-2-yl]-2,3-dihydro-1H-isoindole-5-carboxamide;
1-(4-amino-6-phenyl-1,3,5-triazin-2-yl)-N-{[2-(trifluoromethyl)phenyl]methyl}-1,2,3,4-tetrahydro-6-quinolinecarboxamide;
1-(4-amino-6-phenyl-1,3,5-triazin-2-yl)-N-{[2-(trifluoromethyl)phenyl]methyl}-2,3-dihydro-1H-indole-5-carboxamide;
N-[(2,4-dichlorophenyl)methyl]-1-[4-(methylamino)-6-phenyl-1,3,5-triazin-2-yl]-1,2,3,4-tetrahydro-5-quinolinecarboxamide;
N-[(2,4-dichlorophenyl)methyl]-1-[4-methyl-6-(methylamino)-1,3,5-triazin-2-yl]-1,2,3,4-tetrahydro-5-quinolinecarboxamide;
1-[4-(methylamino)-6-phenyl-1,3,5-triazin-2-yl]-N-({[2-(trifluoromethyl)oxy]phenyl}methyl)-1,2,3,4-tetrahydro-7-quinolinecarboxamide;
1-[4-(4-chlorophenyl)-6-(methylamino)-1,3,5-triazin-2-yl]-N-[(2,4-dichlorophenyl)methyl]-1,2,3,4-tetrahydro-6-quinolinecarboxamide;
N-[(2,4-dichlorophenyl)methyl]-1-[4-(2-fluorophenyl)-6-(methylamino)-1,3,5-triazin-2-yl]-1,2,3,4-tetrahydro-6-quinolinecarboxamide;
N-[(2,4-dichlorophenyl)methyl]-1-{4-(methylamino)-6-[4-(methyloxy)phenyl]-1,3,5-triazin-2-yl}-1,2,3,4-tetrahydro-6-quinolinecarboxamide;
N-[(2,4-dichlorophenyl)methyl]-1-[4-(methylamino)-6-(4-pyridinyl)-1,3,5-triazin-2-yl]-1,2,3,4-tetrahydro-6-quinolinecarboxamide;
N-[(2,4-dichlorophenyl)methyl]-1-[4-(2,4-difluorophenyl)-6-(methylamino)-1,3,5-triazin-2-yl]-1,2,3,4-tetrahydro-6-quinolinecarboxamide;
2-(4-amino-6-methyl-1,3,5-triazin-2-yl)-N-({2-[(trifluoromethyl)oxy]phenyl}methyl)-2,3-dihydro-1H-isoindole-5-carboxamide;
2-(4-amino-6-methyl-1,3,5-triazin-2-yl)-N-({4-(1-pyrrolidinyl)-2-[(trifluoromethyl)oxy]phenyl}methyl)-2,3-dihydro-1H-isoindole-5-carboxamide;
2-(4-amino-6-methyl-1,3,5-triazin-2-yl)-N-{[2-chloro-4-(dimethylamino)phenyl]methyl}-2,3-dihydro-1H-isoindole-5-carboxamide;
2-(4-amino-6-methyl-1,3,5-triazin-2-yl)-N-{[4-chloro-2-(trifluoromethyl)phenyl]methyl}-2,3-dihydro-1H-isoindole-5-carboxamide;
N-({2-chloro-4-[(methylsulfonyl)amino]phenyl}methyl)-1-[4-methyl-6-(methylamino)-1,3,5-triazin-2-yl]-2,3-dihydro-1H-indole-5-carboxamide; and
1-(4-amino-6-methyl-1,3,5-triazin-2-yl)-N-({2-[(trifluoromethyl)oxy]phenyl}methyl)-2,3-dihydro-1H-indole-5-carboxamide;
   or a pharmaceutically acceptable salt thereof.

Compound Preparation The compounds according to Formula I are prepared using conventional organic syntheses. Suitable synthetic routes are depicted below in the following general reaction schemes. All functional groups are as defined in Formula I unless otherwise defined. Starting materials and reagents depicted below in the general reaction schemes are commercially available or can be made from commercially available starting materials using methods known by those skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

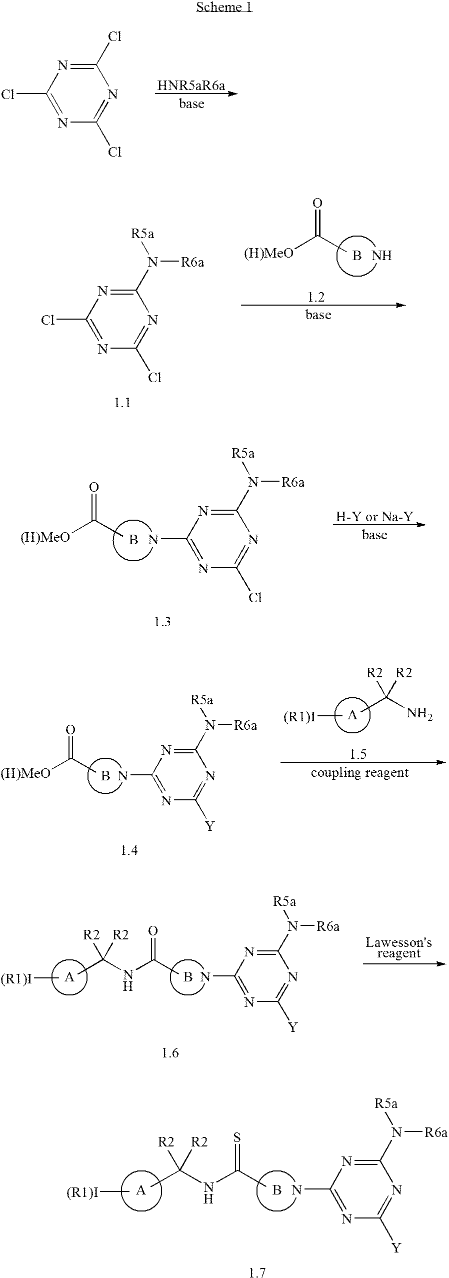

Scheme 1 represents a general reaction scheme for preparing certain compounds according to Formula I (depicted as compounds 1.6 and 1.7). Treatment of cyanuric chloride (commercially available) with amine HNR5aR6a (commercially available or made from commercially available starting materials using methods known to those skilled in the art) and a base (such as NaOH) with a solvent (such as MeCN and water) at temperatures between 0° C. to 50° C. provides intermediate 1.1. Treatment of intermediate 1.1 with amino-methyl carboxylate/amino-carboxylic acid 1.2 and base (such as NaOH) in a solvent (such as MeCN and water) at temperatures between 25° C. to 80° C. provides intermediate 1.3. Treatment of intermediate 1.3 with the desired amine, thiol, or sodium alkoxide and base (such as NaOH) in a solvent (such as MeCN and water) at temperatures between 25° C. to 80° C. provides intermediate 1.4 wherein Y is OH, R12, or NR5bR6b). When intermediate 1.4 contains a methyl ester moiety, hydrolysis with base (such as NaOH) in a solvent (such as NAPnH) at temperatures between 25° C. to 80° C. provides the carboxylic acid intermediate 1.4 necessary for the subsequent coupling step. Treatment of intermediate 1.4 with amine 1.5 (commercially available or made from commercially available starting materials using methods known to those skilled in the art) and a coupling reagent (such as EDCl) in a solvent (such as DMF) at temperatures between 25° C. to 80° C. provides compounds according to Formula I (depicted as compound 1.6) wherein Y is OH, R12, or NR5bR6b. Treatment of compound 1.6 with a thiolating agent (such as Lawesson's Reagent) in a solvent (such as toluene) at temperatures between 25° C. to 80° C. provides compounds according to Formula I (depicted as compound 1.7) wherein Y is OH, R12, or NR5bR6b.

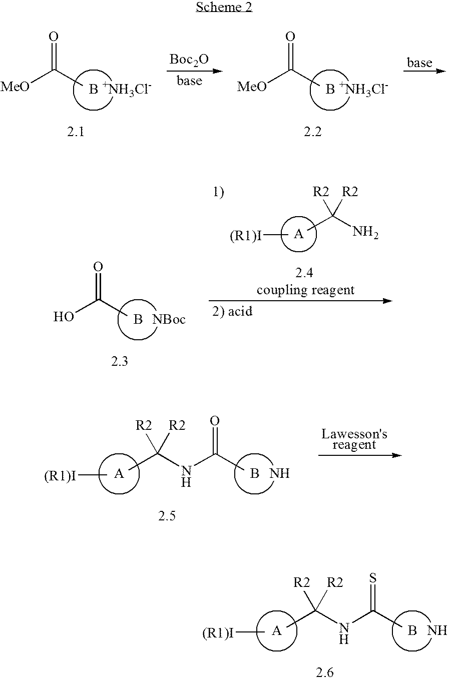

Scheme 2 represents a general reaction scheme for preparing intermediates 2.5 and 2.6. Treatment of compound 2.1 (commercially available or made from commercially available starting materials using methods known to those skilled in the art) with BOC anhydride in the presence of base (such as NaOH) in a solvent (such as 1,4-dioxane and water) at temperatures between 0° C. and 50° C. provides intermediate 2.2. Treatment of intermediate 2.2 with base (such as NaOH) in a solvent (such as MeOH) at temperatures between 25° C. and 80° C. provides intermediate 2.3. Treatment of intermediate 2.3 with amine 2.4 (commercially available or made from commercially available starting materials using methods known to those skilled in the art) and a coupling reagent (such as EDCl) in a solvent (such as DMF) at temperatures between 25° C. and 80° C. provides intermediate 2.5. Treatment of intermediate 2.5 with a thiolating agent (such as Lawesson's Reagent) in a solvent (such as toluene) at temperatures between 25° C. and 80° C. provides intermediate 2.6.

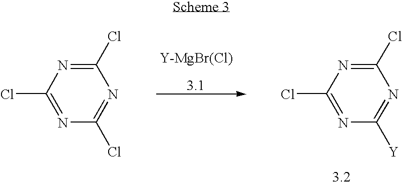

Scheme 3 represents a general reaction scheme for preparing intermediate 3.2. Treatment of cyanuric chloride (commercially available) with Grignard reagent 3.1 (commercially available or made from commercially available starting materials using methods known to those skilled in the art) in a solvent (such as THF) at temperatures between 0° C. and 50° C. provides intermediate 3.2.

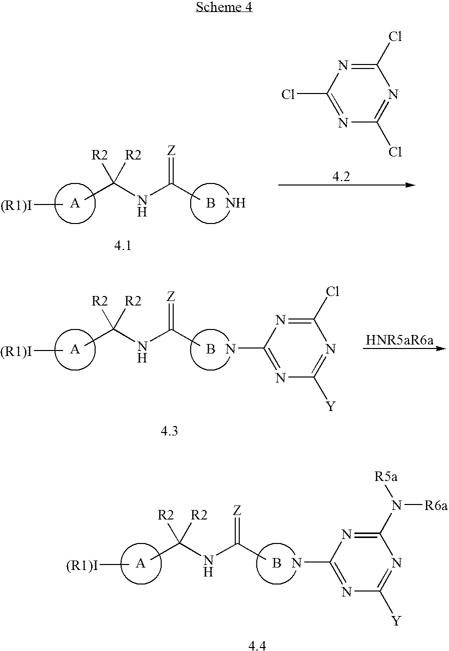

Scheme 4 represents a general reaction scheme for preparing certain compounds according to Formula I. Treatment of intermediate 4.1 (depicted above as intermediate 2.5 or 2.6) with intermediate 4.2 (depicted above as intermediate 3.2) in a solvent (such as MeCN) at temperatures between 85° C. and 170° C. in a microwave reactor provides intermediate 4.3. Treatment of intermediate 4.3 with amine HNR5aR6a (commercially available or made from commercially available starting materials using methods known to those skilled in the art) in a solvent (such as MeCN) at temperatures between 25° C. and 80° C. provides compounds according to Formula I wherein Y is R10 or R11.

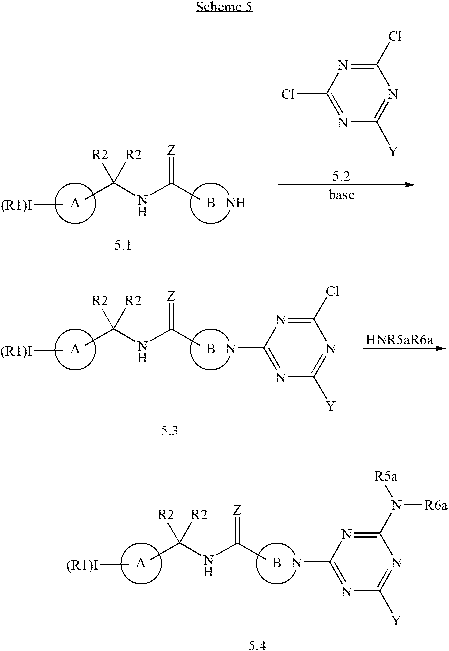

Scheme 5 represents a general reaction scheme for preparing certain compounds according to Formula I. Treatment of intermediate 5.1 (depicted above as intermediate 2.5 or 2.6) with intermediate 4.2 (depicted above as intermediate 3.2), in the presence of a base (such as DIEA) in a solvent (such as MeCN) at temperatures between 25° C. and 80° C. provides intermediate 5.3. Treatment of intermediate 5.3 with amine HNR5aR6a (commercially available or made from commercially available starting materials using methods known to those skilled in the art) in a solvent (such as MeCN) at temperatures between 25° C. and 80° C. provides compounds according to Formula I wherein Y is R7, R8, or R9.

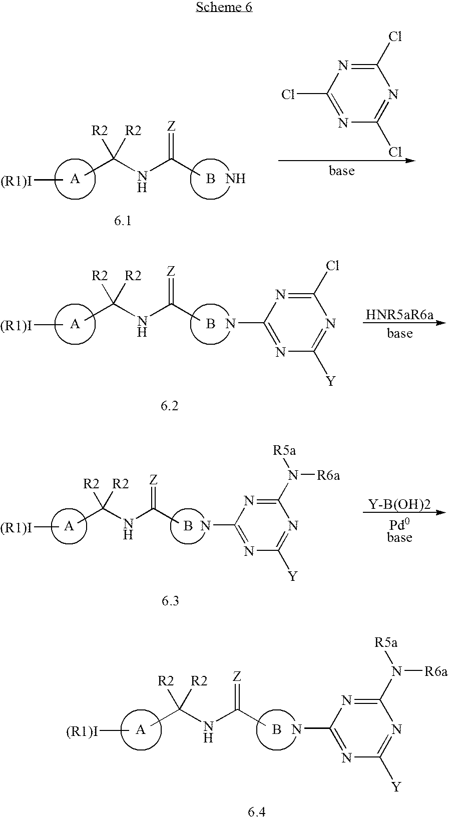

Scheme 6 represents a general reaction scheme for preparing certain compounds according to Formula I. Treatment of intermediate 6.1 (depicted above as intermediate 2.5 or 2.6) with cyanuric chloride (commercially available) in the presence of base (such as DIEA) in a solvent (such as MeCN) at temperatures between 0° C. and 50° C. provides intermediate 6.2. Treatment of intermediate 6.2 with amine HNR5aR6a (commercially available or made from commercially available starting materials using methods known to those skilled in the art) in a solvent (such as MeCN) at temperatures between 25° C. and 80° C. provides intermediate 6.3. Treatment of intermediate 6.3 with the appropriate aryl/heteroaryl boronic acid (commercially available or made from commercially available starting materials using methods known to those skilled in the art) and palladium (0) (such as Pd(PPh₃)₄) with base such as Na₂CO₃ in solvent such as 1,4-dioxane and water at temperatures between 85° C. and 170° C. in a microwave reactor provides compounds according to Formula I wherein Y is R10 or R11.

### Examples

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

### Intermediate 1

### 2,4-dichloro-6-methyl-1,3,5-triazine

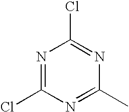

Methyl magnesium bromide (3M in ethyl ether, 9 ml, 27 mmol) was added dropwise over 10 min to a cooled (0° C.) solution of 2,4,6-trichloro-1,3,5-triazine (5 g, 27 mmol) in tetrahydrofuran (270 ml). The reaction mixture was stirred at ambient temperature for 20 hours and was then quenched with saturated NH₄Cl (aq) (100 mL). The aqueous layer was extracted using dichloromethane (3×50 mL), and the combined organic layers were dried over Na₂SO₄, filtered, and concentrated to remove the most of solvent. Celite was then added to the solution and the resulting suspension was concentrated to dryness under vacuum and purified using silica gel chromatography (120 g SiO₂ gel column; solvent dichloromethane/hexane from 0 to 30%) to give 2,4-dichloro-6-methyl-1,3,5-triazine (1.38 g of 90% purity, 7.57 mmol, 28%). MS (ES) m/e 164 [M+H]+.

### Intermediate 2

### 1-{4-bromo-2-[(trifluoromethyl)oxy]phenyl}methanamine

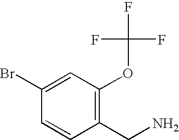

### Step 1: 4-bromo-2-[(trifluoromethyl)oxy]benzaldehyde

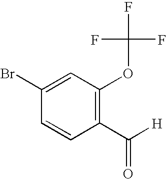

5-bromo-2-iodophenyl trifluoromethyl ether (500 mg, 1.37 mmol) was dissolved in 10 mL of anhydrous THF and cooled to −70° C. Then, n-butyllithium (0.55 mL of a 2.5 M solution, 1.37 mmol) was added dropwise over the course of 30 minutes. DMF (0.19 mL, 2.74 mmol) was added and the reaction was stirred for 30 minutes at −70° C. and then allowed to warm to 0° C. and stir for three hours. The reaction was quenched with 5 mL of saturated ammonium chloride solution and extracted with ethyl acetate. The organic layer was washed with water, dried over magnesium sulfate, filtered, and concentrated to provide 4-bromo-2-[(trifluoromethyl)oxy]benzaldehyde (100 mg, 0.37 mmol, 27%) as a yellow solid. ¹H NMR (400 MHz, DMSO-D6) □10.1 (s, 1H), 7.9 (s, 3H)

### Step 2: 1-{4-bromo-2-[(trifluoromethyl)oxy]phenyl}methanamine

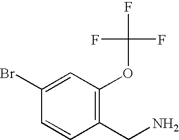

4-bromo-2-[(trifluoromethyl)oxy]benzaldehyde (3 g, 11.2 mmol) was dissolved in 100 mL of a 5M solution of ammonia in methanol and stirred overnight, after which the reaction mixture was treated with sodium borohydride (858 mg, 22.5 mmol) and stirred at room temperature for four days. The reaction was quenched by the addition of 20 mL of water and stirred for 30 minutes. The volatiles were removed and the residue was extracted with methylene chloride (3×20 mL). The methylene chloride was evaporated to give a yellow oil which was purified by preparative HPLC to provide the TFA salt of 1-{4-bromo-2-[(trifluoromethyl)oxy]phenyl}methanamine (900 mg, 3.3 mmol, 29%) as a white solid. MS (ES) m/e 270, 272 [M+H]⁺. ¹H NMR (400 MHz, DMSO-D6) □8.5 (bs, 2H), 7.8 (d, 1H), 7.7 (s, 1H), 7.6 (d, 1H), 4.1 (bs, 2H)

### Intermediate 3

### [4-(aminomethyl)-3-chlorophenyl]dimethylamine

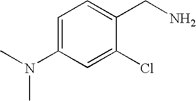

### Step 1: 2-chloro-4-(dimethylamino)benzonitrile

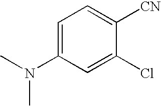

A mixture of 4-amino-2-chlorobenzonitrile (1 g, 6.55 mmol), iodomethane (7.44 g, 52.4 mmol) and cesium carbonate (4.27 g, 13.11 mmol) in acetonitrile (10 mL) were heated to 100° C. in a sealed-tube. After stirring overnight, water (15 mL) was added to the mixture, followed by EtOAc (25 mL). The organic layer was separated and the aqueous re-extracted with EtOAc (25 mL). The organics were dried over Na₂SO₄ and evacuated. Column chromatography (EtOAc/Hexanes=0-40%) afforded the desired product as a white solid (0.76 g, 64%). MS (ES+): m/e 180.8 [M+H]⁺.

### Step 2: [4-(aminomethyl)-3-chlorophenyl]dimethylamine

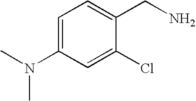

To a solution of 2-chloro-4-(dimethylamino)benzonitrile (0.76 g, 4.21 mmol) in THF (5 mL) was added 1M LiAlH₄/I₂ in THF (8.41 ml, 8.41 mmol, as prepared in Step 2 of Intermediate 3). The mixture was stirred for 10 min, at which time LCMS indicated the formation of the desired product. Water (15 mL) was added to the mixture, which was acidified to pH=2 with 6N HCl. The aqueous layer was separated and washed with Et₂O (2×25 mL). The aqueous layer was then basified with 6N NaOH, and extracted with Et₂O (3×30 mL). The organics were dried over Na₂SO₄. The ether layer was evacuated to ⅓ volume at which time 8 mL of 1M HCl in ether solution was added. After stirring for 10 minutes, solids precipitated from solution. The solids were filtered, washed with ether and dried to afford the title compound (1.0 g, 92%) as a light yellow solid. MS (ES+): m/e 185.0 [M+H]⁺.

### Intermediate 4

### N-[4-(aminomethyl)-3-chlorophenyl]methanesulfonamide

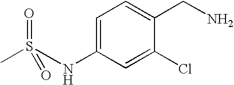

### Step 1: N-(3-chloro-4-cyanophenyl)methanesulfonamide

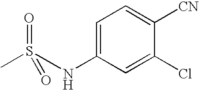

To a solution of 4-amino-2-chlorobenzonitrile (5 g, 32.8 mmol) and pyridine (3.18 mL, 39.3 mmol) in Dichloromethane (DCM) (75 mL) was added methanesulfonyl chloride (4.50 g, 39.3 mmol) slowly at 0° C. under the N₂. After addition, the mixture was warmed to room temperature and stirred overnight. The reaction mixture was treated with 50 mL of 2N NaOH. The layers were separated. The aqueous layer was acidified with conc.HCl to pH=2, which resulted in the precipitation of product. The solids were filtered, washed with water, and dried to give the desired product as a white solid (7.0 g, 93%). MS (ES+): m/e 230.8 [M+H]⁺.

### Step 2: N-[4-(aminomethyl)-3-chlorophenyl]methanesulfonamide

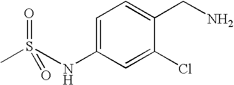

In a three-neck round-bottom flask containing a 0° C. solution of LiAlH₄ (2M in THF) (26.0 mL, 52.0 mmol) was added iodine (6.60 g, 26.0 mmol) in THF (20 mL) dropwise under N₂. After addition, the mixture was stirred for 30 min at 0° C. N-(3-chloro-4-cyanophenyl)methanesulfonamide (4 g, 17.34 mmol) in THF (20 mL) was then added dropwise. After addition, the reaction mixture was warmed to room temperature and stirred for 1 h, during which time precipitate crashed out of the solution. The reaction mixture was filtered, and the cake washed with cold THF. The cake was carefully transferred to a beaker which contained 60 mL of THF. The mixture was acidified to pH=2 with 6N HCl with constant stirring at 0° C. The layers were separated and the aqueous layer was washed with DCM (30 mL). The aqueous layer was concentrated by rotary evaporation. The resulting solids were washed with cold MeOH to give a yellow solid. The mother liquid was subjected to the same workup (2×). The combined crops were collected and dried to give the title compound as the HCl salt (4.2 g, 89%). MS (ES+): m/e 234.7 [M+H]⁺.

### Intermediate 5

### 2-(4-amino-6-methyl-1,3,5-triazin-2-yl)-2,3-dihydro-1H-isoindole-5-carboxylic acid

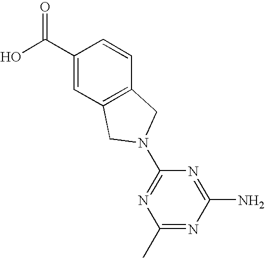

To a mixture of methyl 2,3-dihydro-1H-isoindole-5-carboxylate (0.400 g, 1.872 mmol) and 2,4-dichloro-6-methyl-1,3,5-triazine (0.307 g, 1.872 mmol) in acetonitrile (15 ml) was added diisopropylethylamine (0.687 ml, 3.93 mmol) dropwise. The reaction was stirred at room temperature for 45 minutes, after which time LCMS indicated the formation of the desired intermediate. Next, ammonium hydroxide (4.00 ml, 33.9 mmol) was added to the reaction, which was sealed and heated to 80° C. overnight. LC/MS indicated the formation of the desired intermediate. The reaction was cooled, and the resulting precipitate filtered and washed with water. The precipitate was then suspended in MeOH (5 mL) and treated with 2.5N NaOH (5 mL). The reaction was heated to 65° C. for 1 hr, at which time LCMS showed the formation of the desired product. The reaction was cooled, and acidified to pH 4 with 3N HCl. The resulting precipitate was vacuum filtered and washed with water and acetonitrile to provide 2-(4-amino-6-methyl-1,3,5-triazin-2-yl)-2,3-dihydro-1H-isoindole-5-carboxylic acid (370 mg, 1.36 mmol, 73%). MS (ES) m/e 272 [M+H]⁺. ¹H NMR (400 MHz, DMSO-D6) □13.0 (bs, 1H), 8.0 (m, 1H), 7.9 (d, 1H), 7.5 (m, 1H), 6.8 (bs, 2H), 4.8 (bs, 2H), 4.7 (bs, 2H), 2.2 (s, 3H)

### Intermediate 6

### 1-[4-methyl-6-(methylamino)-1,3,5-triazin-2-yl]-2,3-dihydro-1H-indole-5-carboxylic acid

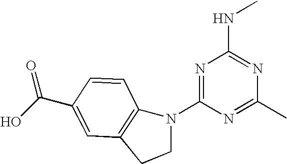

Intermediate 6 was prepared using the general procedure described above in Intermediate 5 substituting methyl 2,3-dihydro-1H-indole-5-carboxylate for methyl 2,3-dihydro-1H-isoindole-5-carboxylate. MS (ES) m/e 286 [M+H]⁺.

### Intermediate 7

### N-({2-[(trifluoromethyl)oxy]phenyl}methyl)-2,3-dihydro-1H-indole-5-carboxamide, trifluoroacetate salt

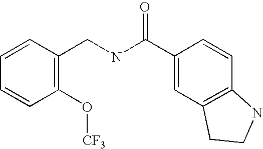

### Step 1: N-({2-[(trifluoromethyl)oxy]phenyl}methyl)-1H-indole-5-carboxamide

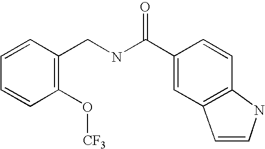

1H-indole-5-carboxylic acid (5 g, 31.0 mmol) and 1-{2-[(trifluoromethyl)oxy]phenyl}methanamine (5.22 ml, 34.1 mmol) were dissolved in dimethylformamide (DMF) (40 mL) at room temperature. Afterwards, triethylamine (4.32 ml, 31.0 mmol) was added and the solution was allowed to stir for several minutes before a separate solution of 1H-1,2,3-benzotriazol-1-yloxy-tris(dimethylamino)-phosphonium hexafluorophosphate (BOP reagent, 13.72 g, 31.0 mmol) dissolved in 15 mL of DMF was delivered to the mixture at room temperature. The reaction was maintained at that temperature for 2.5 hours, before it was determined to be complete by LC/MS. The crude mixture was slowly poured into a vigorously stirring solution (550 mL) of saturated sodium bicarbonate and water (1:1) at room temperature which resulted in the precipitation of the desired product as an off-white solid. The mixture was allowed to stir for 18 hr, before the solid was recovered by vacuum filtration and dried for 24 hours under vacuum at 65° C. to give N-({2-[(trifluoromethyl)oxy]phenyl}methyl)-1H-indole-5-carboxamide (9.44 g, 28.0 mmol, 90% yield). MS (ES) m/e 335 [M+H]+.

### Step 2: N-({2-[(trifluoromethyl)oxy]phenyl}methyl)-2,3-dihydro-1H-indole-5-carboxamide, trifluoroacetate salt

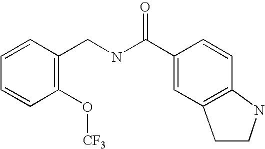

A solution of N-({2-[(trifluoromethyl)oxy]phenyl}methyl)-1H-indole-5-carboxamide (2 g, 5.98 mmol) dissolved in trifluoroacetic acid (28.7 ml, 372 mmol) was prepared in a 25 mL round bottom flask equipped with a magnetic stir bar under argon. Triethylsilane (28.6 ml, 179 mmol) was delivered to the flask at room temperature. The mixture was allowed to stir for 18 hr, before the reaction was determined to be complete by LC/MS. The reaction mixture was concentrated under vacuum to provide N-({2-[(trifluoromethyl)oxy]phenyl}methyl)-2,3-dihydro-1H-indole-5-carboxamide, trifluoroacetate salt as a crude orange oil which was carried on to the next step without further purification. MS (ES) m/e 335 [M+H]+.

### Example 1

### N-[(2,4-dichlorophenyl)methyl]-2-[4-(methylamino)-6-(4-methyl-1-piperazinyl)-1,3,5-triazin-2-yl]-2,3-dihydro-1H-isoindole-5-carboxamide

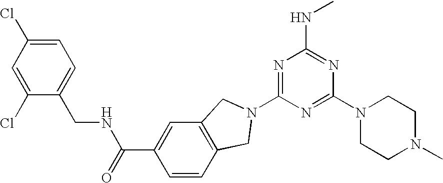

### Step 1: Preparation of 4,6-dichloro-N-methyl-1,3,5-triazin-2-amine

To a 0° C. mixture of cyanuric chloride (0.50 g, 2.73 mmol) in CH₃CN/H₂O (1:1, 4.5 mL) was added methyl amine (2M THF, 1.37 mL, 2.73 mmol) dropwise. The reaction mixture was adjusted to pH 10 using 1N NaOH. The pH was maintained at 10 for 30 minutes. LCMS was used to monitor the reaction. Without workup or purification, the product was used directly in the next step in the same reaction vessel.

### Step 2: Preparation of methyl 2-[4-chloro-6-(methylamino)-1,3,5-triazin-2-yl]-2,3-dihydro-1H-isoindole-5-carboxylate

To the reaction mixture from Step 1 was added methyl isoindoline-5-carboxylate hydrochloride (0.58 g, 2.73 mmol). The reaction was stirred at room temperature for 1 hour while the pH was maintained at 10 using 1N NaOH. LCMS was used to monitor the reaction. Without workup or purification, the product was used directly in the next step in the same reaction vessel

### Step 3: Preparation of methyl 2-[4-(methylamino)-6-(4-methyl-1-piperazinyl)-1,3,5-triazin-2-yl]-2,3-dihydro-1H-isoindole-5-carboxylate

To the reaction mixture from Step 2 was added 1-methyl-piperazine (1.51 mL, 13.7 mmol). The reaction was heated to 80° C. and stirred for 1 hour, at which time LCMS indicated complete conversion to product. The solvent was evacuated and the crude material was used directly in the next step.

### Step 4: Preparation of 2-[4-(methylamino)-6-(4-methyl-1-piperazinyl)-1,3,5-triazin-2-yl]-2,3-dihydro-1H-isoindole-5-carboxylic acid

A solution of the crude methyl ester from Step 3 was dissolved in MeOH (5 mL) and treated with 1N NaOH (5 mL). The reaction was heated to 50° C. for 1 hour, at which time LCMS indicated complete conversion to the acid. The reaction was cooled, evacuated, and then purified directly by RP-HPLC (gradient; 10-50% CH₃CN:H₂O (0.1% TFA)), to afford the title compound (534 mg, 53%, 4 steps). MS (ES+) m/e 370.0 [M+H]⁺.

### Step 5: Preparation of N-[(2,4-dichlorophenyl)methyl]-2-[4-(methylamino)-6-(4-methyl-1-piperazinyl)-1,3,5-triazin-2-yl]-2,3-dihydro-1H-isoindole-5-carboxamide

To a solution of carboxylic acid (50.0 mg, 0.14 mmol) and 2,4-dichlorobenzylamine (18.0 □L, 0.14 mmol) in DMF (2 mL) was added Hunig's Base (41.0 □L, 0.24 mmol) followed by BOP (65.0 mg, 0.24 mmol). The reaction was stirred at room temperature for 3 hours, at which time the reaction was deemed complete by LCMS. The reaction mixture was purified directly by RP-HPLC (gradient 20-60% CH₃CN:H₂O (0.1% TFA)), to afford the title compound (27 mg, 38%). MS (ES+) m/e 526.9 [M+H]⁺.

### Example 2

### 2-[4-(methylamino)-6-(4-methyl-1-piperazinyl)-1,3,5-triazin-2-yl]-N-{[2-(trifluoromethyl)phenyl]methyl}-2,3-dihydro-1H-isoindole-5-carboxamide trifluoroacetate

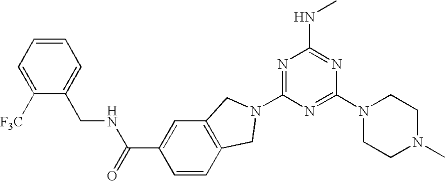

Example 2 was prepared using the general procedure described above in Example 1 substituting the appropriate starting materials. MS (ES+): m/e 526.9 [M+H]⁺

### Example 3

### 2-[4-(methylamino)-6-(4-methyl-1-piperazinyl)-1,3,5-triazin-2-yl]-N-({2-[(trifluoromethyl)oxy]phenyl}methyl)-2,3-dihydro-1H-isoindole-5-carboxamide trifluoroacetate

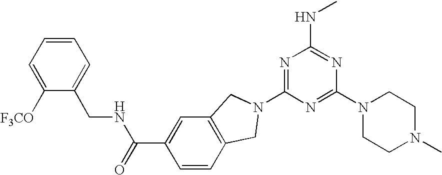

Example 3 was prepared using the general procedure described above in Example 1 substituting the appropriate starting materials. MS (ES+): m/e 542.9 [M+H]⁺

### Example 4

### N-{[4-chloro-2-(trifluoromethyl)phenyl]methyl}-2-[4-(methylamino)-6-(4-methyl-1-piperazinyl)-1,3,5-triazin-2-yl]-2,3-dihydro-1H-isoindole-5-carboxamide trifluoroacetate

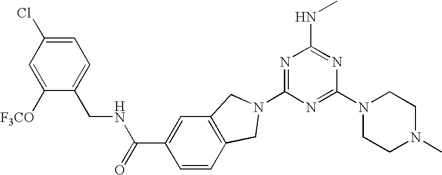

Example 4 was prepared using the general procedure described above in Example 1 substituting the appropriate starting materials. MS (ES+): m/e 560.9 [M+H]⁺

### Example 5

### 2-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-isoquinolinecarboxamide

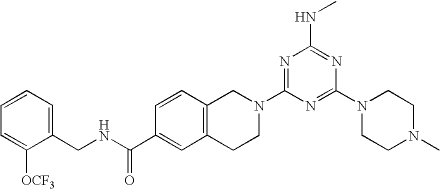

Example 5 was prepared using the general procedure described above in steps 1 to 5 of Example 1 substituting methyl 1,2,3,4-tetrahydroisoquinoline-6-carboxylate hydrochloride for methyl isoindoline-5-carboxylate hydrochloride in step 2 and 2-(trifluoromethoxy)benzylamine for 2,4-dichlorobenzylamine in step 5. MS (ES+) m/e 556.9 [M+H]⁺.

### Example 6

### 2-[4-(methylamino)-6-(4-methyl-1-piperazinyl)-1,3,5-triazin-2-yl]-N-{[2-(trifluoromethyl)phenyl]methyl}-1,2,3,4-tetrahydro-6-isoquinolinecarboxamide

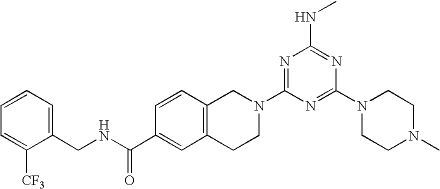

Example 6 was prepared using the general procedure described above in Example 5 substituting the appropriate benzyl amine for 2-(trifluoromethoxy)benzylamine. MS (ES+): m/e 540.9 [M+H]⁺

### Example 7

### 2-[4-(methylamino)-6-(4-methyl-1-piperazinyl)-1,3,5-triazin-2-yl]-N-{[4-(trifluoromethyl)phenyl]methyl}-1,2,3,4-tetrahydro-6-isoquinolinecarboxamide

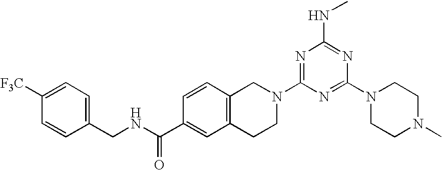

Example 7 was prepared using the general procedure described above in Example 6 substituting the appropriate benzyl amine for 2-(trifluoromethoxy)benzylamine. MS (ES+): m/e 540.9 [M+H]⁺

### Example 8

### N-[(2,5-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide

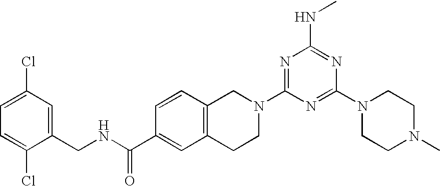

Example 8 was prepared using the general procedure described above in Example 6 substituting the appropriate benzyl amine for 2-(trifluoromethoxy)benzylamine. MS (ES+): m/e 540.8 [M+H]⁺

### Example 9

### 4-{[({2-[4-(methylamino)-6-(4-methyl-1-piperazinyl)-1,3,5-triazin-2-yl]-1,2,3,4-tetrahydro-6-isoquinolinyl}carbonyl)amino]methyl}benzoic acid

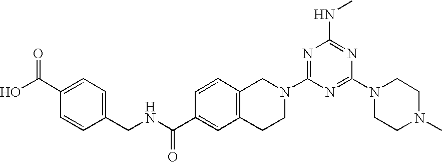

Example 9 was prepared using the general procedure described above in Example 6 substituting the appropriate benzyl amine for 2-(trifluoromethoxy)benzylamine. MS (ES+): m/e 517.0 [M+H]⁺

### Example 10

### N-[(2,4-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide

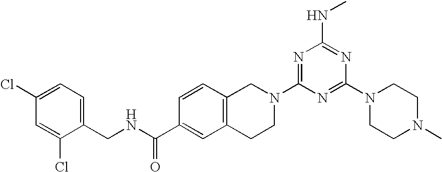

Example 10 was prepared using the general procedure described above in Example 6 substituting the appropriate benzyl amine for 2-(trifluoromethoxy)benzylamine. MS (ES+): m/e 540.9 [M+H]⁺

### Example 11

### N-{[4-chloro-2-(trifluoromethyl)phenyl]methyl}-2-[4-(methylamino)-6-(4-methyl-1-piperazinyl)-1,3,5-triazin-2-yl]-1,2,3,4-tetrahydro-6-isoquinolinecarboxamide

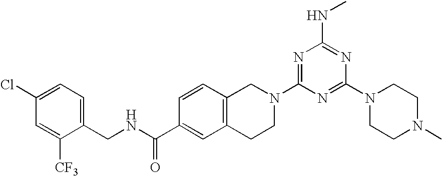

Example 11 was prepared using the general procedure described above in Example 6 substituting the appropriate benzyl amine for 2-(trifluoromethoxy)benzylamine. MS (ES+): m/e 575.0 [M+H]⁺

### Example 12

### 1-[4-(methylamino)-6-(4-methyl-1-piperazinyl)-1,3,5-triazin-2-yl]-N-{[2-(trifluoromethyl)phenyl]methyl}-1,2,3,4-tetrahydro-6-quinolinecarboxamide

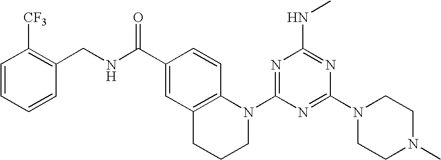

Example 12 was prepared using the general procedure described above in Example 1 substituting 1,2,3,4-tetrahydro-6-quinolinecarboxylic acid for methyl isoindoline-5-carboxylate hydrochloride, omitting step 4, and substituting 2-(trifluoromethyl)benzylamine for 2,4-dichlorobenzylamine in step 5. MS (ES+) 542.2 m/e [M+H]⁺.

### Example 13

### 1-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-quinolinecarboxamide

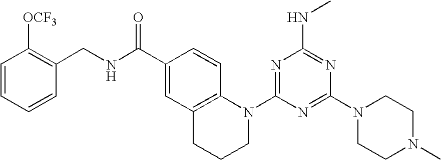

Example 13 was prepared using the general procedure described above in Example 12 substituting the appropriate benzyl amine for 2-(trifluoromethyl)benzylamine. MS (ES+): m/e 556.8 [M+H]⁺

### Example 14

### N-[(2,4-dichlorophenyl)methyl]-1-[4-(methylamino)-6-(4-methyl-1-piperazinyl)-1,3,5-triazin-2-yl]-1,2,3,4-tetrahydro-6-quinolinecarboxamide

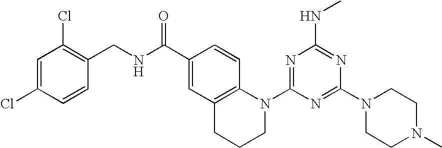

Example 14 was prepared using the general procedure described above in Example 12 substituting the appropriate benzyl amine for 2-(trifluoromethyl)benzylamine. MS (ES+): m/e 540.8 [M+H]⁺

### Example 15

### N-[(2,5-dichlorophenyl)methyl]-1-[4-(methylamino)-6-(4-methyl-1-piperazinyl)-1,3,5-triazin-2-yl]-1,2,3,4-tetrahydro-6-quinolinecarboxamide

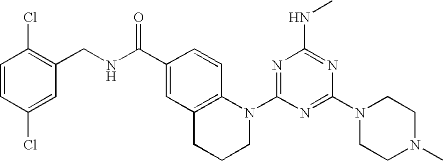

Example 15 was prepared using the general procedure described above in Example 12 substituting the appropriate benzyl amine for 2-(trifluoromethyl)benzylamine. MS (ES+): m/e 540.8 [M+H]⁺

### Example 16

### 1-[4-(methylamino)-6-(4-methyl-1-piperazinyl)-1,3,5-triazin-2-yl]-N-{[4-(trifluoromethyl)phenyl]methyl}-1,2,3,4-tetrahydro-6-quinolinecarboxamide

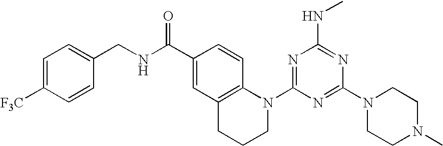

Example 16 was prepared using the general procedure described above in Example 12 substituting the appropriate benzyl amine for 2-(trifluoromethyl)benzylamine. MS (ES+): m/e 540.9 [M+H]⁺

### Example 17

### 1-[4-(methylamino)-6-(4-methyl-1-piperazinyl)-1,3,5-triazin-2-yl]-N-{[2-(methyloxy)phenyl]methyl}-1,2,3,4-tetrahydro-6-quinolinecarboxamide

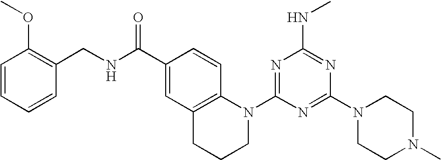

Example 17 was prepared using the general procedure described above in Example 12 substituting the appropriate benzyl amine for 2-(trifluoromethyl)benzylamine. MS (ES+): m/e 502.9 [M+H]⁺

### Example 18

### N-[(3,4-difluorophenyl)methyl]-1-[4-(methylamino)-6-(4-methyl-1-piperazinyl)-1,3,5-triazin-2-yl]-1,2,3,4-tetrahydro-6-quinolinecarboxamide

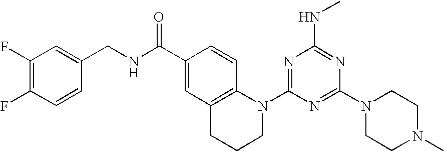

Example 18 was prepared using the general procedure described above in Example 12 substituting the appropriate benzyl amine for 2-(trifluoromethyl)benzylamine. MS (ES+): m/e 508.9 [M+H]⁺

### Example 19

### 1-[4-(methylamino)-6-(4-methyl-1-piperazinyl)-1,3,5-triazin-2-yl]-N-{[2-(trifluoromethyl)phenyl]methyl}-2,3-dihydro-1H-indole-5-carboxamide

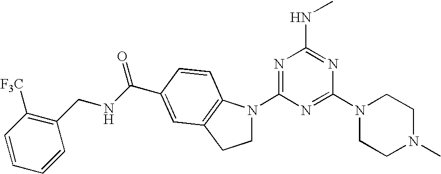

Example 19 was prepared using the general procedure described above in Example 1 substituting 2,3-dihydro-1H-indole-5-carboxylic acid for methyl isoindoline-5-carboxylate hydrochloride, omitting step 4, and substituting 2-(trifluoromethyl)benzylamine for 2,4-dichlorobenzylamine in step 5. MS (ES+) m/e 527.4 [M+1]⁺

### Example 20

### 1-[4-(methylamino)-6-(4-methyl-1-piperazinyl)-1,3,5-triazin-2-yl]-N-({2-[(trifluoromethyl)oxy]phenyl}methyl)-2,3-dihydro-1H-indole-5-carboxamide trifluoroacetate

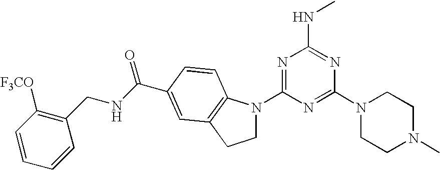

Example 20 was prepared using the general procedure described above in Example 19 substituting the appropriate benzyl amine for 2-(trifluoromethyl)benzylamine. MS (ES+): m/e 543.0 [M+H]⁺

### Example 21

### N-[(2,4-dichlorophenyl)methyl]-1-[4-(methylamino)-6-(4-methyl-1-piperazinyl)-1,3,5-triazin-2-yl]-2,3-dihydro-1H-indole-5-carboxamide trifluoroacetate

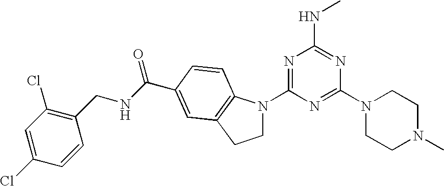

Example 21 was prepared using the general procedure described above in Example 19 substituting the appropriate benzyl amine for 2-(trifluoromethyl)benzylamine. MS (ES+): m/e 526.9 [M+H]⁺

### Example 22

### N-{[4-chloro-2-(trifluoromethyl)phenyl]methyl}-1-[4-(methylamino)-6-(4-methyl-1-piperazinyl)-1,3,5-triazin-2-yl]-2,3-dihydro-1H-indole-5-carboxamide trifluoroacetate

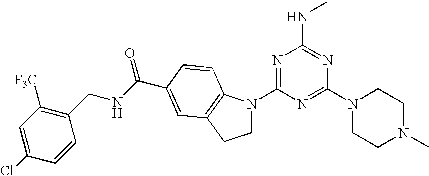

Example 22 was prepared using the general procedure described above in Example 19 substituting the appropriate benzyl amine for 2-(trifluoromethyl)benzylamine. MS (ES+): m/e 560.9 [M+H]⁺

### Example 23

### N-({[2-(trifluoromethyl)oxy]phenyl}methyl)-1,2,3,4-tetrahydro-6-isoquinolinecarboxamide

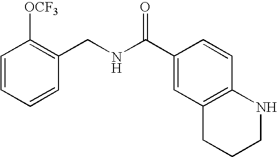

### Step 1: Preparation of 2-(1,1-dimethylethyl) 6-methyl 3,4-dihydro-2,6(1H)-isoquinoline-dicarboxylate

To a solution of methyl 1,2,3,4-tetrahydroisoquinoline-6-carboxylate hydrochloride (0.30 g, 1.32 mmol) in dioxane/H₂O (2:1, 5 mL) was added 1N NaOH (1.97 mL, 1.97 mmol) followed by Boc₂O (0.43 g, 1.97 mmol). The reaction was stirred at room temperature for 1 hour, at which time LCMS indicated complete conversion to product. The reaction was poured into H₂O (10 mL) and extracted with CH₂Cl₂ (3×10 mL). The organics were dried (Na₂SO₄) and evacuated. The crude carbamate was used directly in the next step.

### Step 2: 2-{[(1,1-dimethylethyl)oxy]carbonyl}-1,2,3,4-tetrahydro-6-isoquinolinecarboxylic acid

To a solution of the crude carbamate from Step 1 in MeOH/THF (1:1, 6 mL) was added 1N NaOH (1.97 mL, 1.97 mmol). The reaction was heated to 70° C. for 1 hour. The reaction was cooled and the MeOH evacuated. The remaining aqueous solution was acidified to pH 4 with 1N HCl. The resulting precipitate was vacuum filtered and dried to afford the desired product (0.34 g, 93%) which was used without further purification. MS (ES+) m/e 222.0 [M−tBu]⁺

### Step 3: N-({2-[(trifluoromethyl)oxy]phenyl}methyl)-1,2,3,4-tetrahydro-6-isoquinolinecarboxamide

To a solution of aforementioned carboxylic acid (0.20 g, 0.72 mmol) in DMF (5 mL) was added DMAP (24.0 mg, 0.22 mmol) followed by 2-(trifluoromethoxy)benzylamine (108 □L, 0.79 mmol) and EDCl (138 mg, 0.72 mmol). After stirring overnight, the reaction was poured into H₂O (10 mL) and extracted with EtOAc (2×10 mL). The organics were dried (Na₂SO₄) and evacuated. The crude material was redissolved in CH₂Cl₂ (2 mL) and treated with TFA (2 mL). After stirring for 30 minutes, the reaction was diluted with CH₂Cl₂ (15 mL) and poured into ice cold 1N NaOH (20 mL). The organics were extracted, dried (Na₂SO₄), and evacuated to afford the title compound (0.15 g, 59%), which was used without further purification. MS (ES+) m/e 335.0 [M+1]⁺

### Example 24

### 2,4-dichloro-6-phenyl-1,3,5-triazine

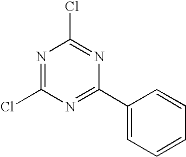

The title compound was prepared via the dropwise addition of phenylmagnesium bromide (1M THF, 10.8 mL, 10.8 mmol) to a 0° C. solution of cyanuric chloride (2.0 g, 10.8 mmol) in THF (50 mL). The reaction was allowed to warm to room temperature and stir for 1.5 hours. Saturated NH₄Cl (20 mL) was added to the reaction, and the organics were extracted with EtOAc (2×50 mL). The organics were dried and evacuated. The crude material was purified by silica gel chromatography (gradient; 0-20% CH₂Cl₂:Hexanes) to afford the title compound as a white solid. MS (ES+) m/e 226.0 [M+H]⁺.

### Example 25

### 2-[4-(methylamino)-6-phenyl-1,3,5-triazin-2-yl]-N-({[2-(trifluoromethyl)oxy]phenyl}methyl)-1,2,3,4-tetrahydro-6-isoquinolinecarboxamide

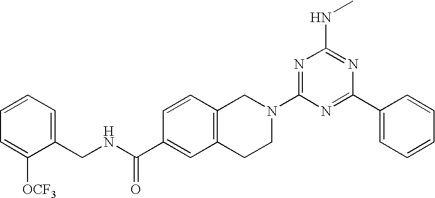

### Step 1: 2-(4-chloro-6-phenyl-1,3,5-triazin-2-yl)-N-({2-[(trifluoromethyl)oxy]phenyl}methyl)-1,2,3,4-tetrahydro-6-isoquinolinecarboxamide

A solution of N-({[2-(trifluoromethyl)oxy]phenyl}methyl)-1,2,3,4-tetrahydro-6-isoquinoline-carboxamide (50.0 mg, 0.14 mmol) as prepared in Example 23 and 2,4-dichloro-6-phenyl-1,3,5-triazine (32.0 mg, 0.14 mmol) as prepared in Example 24 in MeCN (3 mL) was heated in the microwave for 15 minutes at 130° C. LCMS indicated the formation of the title compound which was used directly in the next step in the same reaction vessel.

### Step 2: 2-[4-(methylamino)-6-phenyl-1,3,5-triazin-2-yl]-N-({2-[(trifluoromethyl)oxy]phenyl}methyl)-1,2,3,4-tetrahydro-6-isoquinolinecarboxamide

To the reaction mixture from Step 1 was added MeNH₂ (2M THF, 0.35 mL, 0.70 mmol). The reaction was heated to 40° C. for 30 minutes, at which time LCMS indicated conversion to the desired product. The reaction was evacuated and purified directly by RP-HPLC (gradient 40-90% CH₃CN:H₂O (0.1% TFA)), to afford the title compound (45 mg, 60%). MS (ES+) m/e 534.9 [M+H]⁺.

### Example 26

### 1-[4-(methylamino)-6-phenyl-1,3,5-triazin-2-yl]-N-{[2-(trifluoromethyl)phenyl]methyl}-1,2,3,4-tetrahydro-6-quinolinecarboxamide

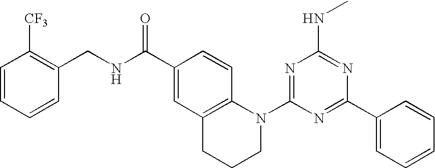

Example 26 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 518.9 [M+H]⁺

### Example 27

### 1-[4-(methylamino)-6-phenyl-1,3,5-triazin-2-yl]-N-{[2-(trifluoromethyl)phenyl]methyl}-2,3-dihydro-1H-indole-5-carboxamide

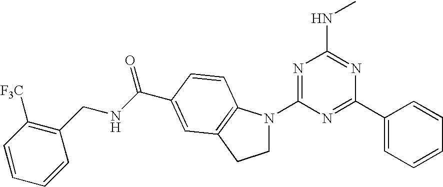

Example 27 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 504.9 [M+H]⁺

### Example 28

### 2-[4-(methylamino)-6-phenyl-1,3,5-triazin-2-yl]-N-{[2-(trifluoromethyl)phenyl]methyl}-1,2,3,4-tetrahydro-6-isoquinolinecarboxamide

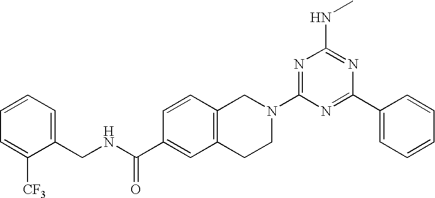

Example 28 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 518.9 [M+H]⁺

### Example 29

### N-[(2,4-dichlorophenyl)methyl]-1-[4-(methylamino)-6-phenyl-1,3,5-triazin-2-yl]-2,3-dihydro-1H-indole-5-carboxamide

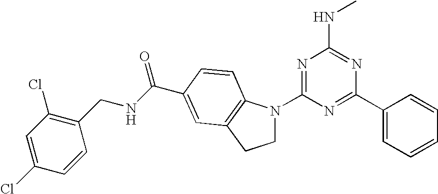

Example 29 was prepared using the general procedure described above in Example 25 substituting the appropriate carboxamide, as prepared using the general procedure described in Example 23, for N-({2-[(trifluoromethyl)oxy]phenyl}methyl)-1,2,3,4-tetrahydro-6-isoquinolinecarboxamide. MS (ES+): m/e 504.8 [M+H]⁺

### Example 30

### N-[(2,4-dichlorophenyl)methyl]-1-[4-(methylamino)-6-phenyl-1,3,5-triazin-2-yl]-1,2,3,4-tetrahydro-6-quinolinecarboxamide

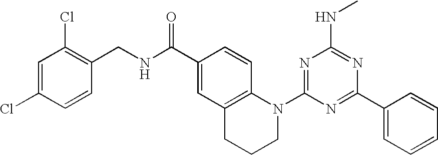

Example 30 was prepared using the general procedure described above in Example 25 substituting the appropriate carboxamide, as prepared using the general procedure described in Example 23, for N-({2-[(trifluoromethyl)oxy]phenyl}methyl)-1,2,3,4-tetrahydro-6-isoquinolinecarboxamide. MS (ES+): m/e 518.8 [M+H]⁺

### Example 31

### 1-[4-(methylamino)-6-phenyl-1,3,5-triazin-2-yl]-N-({2-[(trifluoromethyl)oxy]phenyl}methyl)-2,3-dihydro-1H-indole-5-carboxamide

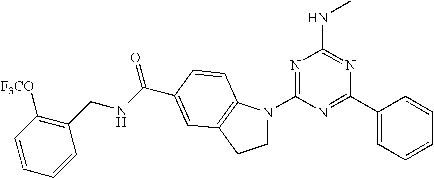

Example 31 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 520.9 [M+H]⁺

### Example 32

### 1-[4-(methylamino)-6-phenyl-1,3,5-triazin-2-yl]-N-({[2-(trifluoromethyl)oxy]phenyl}methyl)-1,2,3,4-tetrahydro-6-quinolinecarboxamide

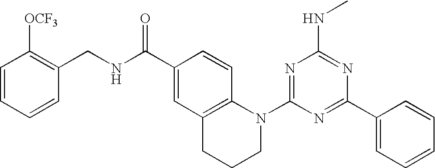

Example 32 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 534.9 [M+H]⁺

### Example 33

### N-[(2,4-dichlorophenyl)methyl]-2-[4-(methylamino)-6-phenyl-1,3,5-triazin-2-yl]-2,3-dihydro-1H-isoindole-5-carboxamide

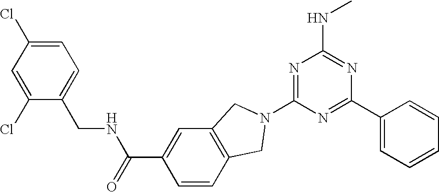

Example 33 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 504.9 [M+H]⁺

### Example 34

### 2-[4-(methylamino)-6-phenyl-1,3,5-triazin-2-yl]-N-{[2-(trifluoromethyl)phenyl]methyl}-2,3-dihydro-1H-isoindole-5-carboxamide

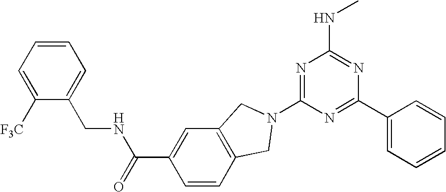

Example 34 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 504.9 [M+H]⁺

### Example 35

### 2-[4-(methylamino)-6-phenyl-1,3,5-triazin-2-yl]-N-({2-[(trifluoromethyl)oxy]phenyl}methyl)-2,3-dihydro-1H-isoindole-5-carboxamide

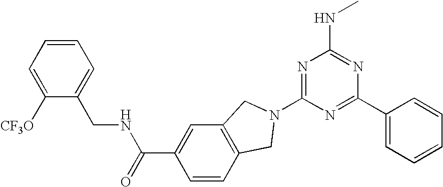

Example 35 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 520.9 [M+H]⁺

### Example 36

### N-[(2,4-dichlorophenyl)methyl]-2-[4-(methylamino)-6-phenyl-1,3,5-triazin-2-yl]-1,2,3,4-tetrahydro-6-isoquinolinecarboxamide trifluoroacetate

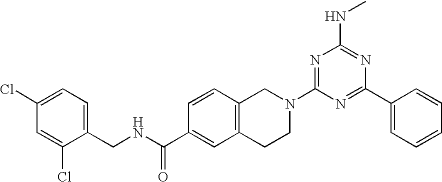

Example 36 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 518.9 [M+H]⁺

### Example 37

### 1-[4-methyl-6-(methylamino)-1,3,5-triazin-2-yl]-N-({2-[(trifluoromethyl)oxy]phenyl}methyl)-2,3-dihydro-1H-indole-5-carboxamide

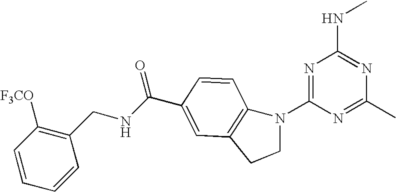

A solution of N-({2-[(trifluoromethyl)oxy]phenyl}methyl)-2,3-dihydro-1H-indole-5-carboxamide (75.0 mg, 0.22 mmol) as prepared similarly to Example 23 (NOTE: 2,3-dihydro-1H-indole-5-carboxylic acid was purchased from commercial sources and directly underwent amidation to afford the benzylcarboxamide) and 2,4-dichloro-6-methyl-1,3,5-triazine (36.0 mg, 0.22 mmol) as prepared similarly to Example 24 in MeCN (5 mL) was treated with Hunig's Base (76.0 □L, 0.44 mmol). The reaction was heated to 40° C. 2 additional equivalents of 2,4-dichloro-6-methyl-1,3,5-triazine were added over the course of 1 hour in order to afford the desired intermediate. Thereafter, the procedure from Step 2 of Example 25 was followed to afford the title compound (50 mg, 52%). MS (ES+) m/e 459.0 [M+H]⁺.

### Example 38

### 2-[4-methyl-6-(methylamino)-1,3,5-triazin-2-yl]-N-({[2-(trifluoromethyl)oxy]phenyl}methyl)-1,2,3,4-tetrahydro-6-isoquinolinecarboxamide

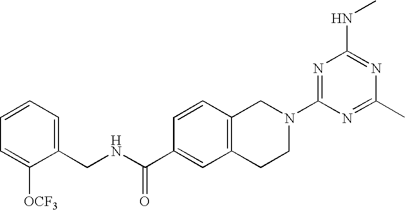

Example 38 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 473.0 [M+H]⁺

### Example 39

### 1-[4-methyl-6-(methylamino)-1,3,5-triazin-2-yl]-N-({[2-(trifluoromethyl)oxy]phenyl}methyl)-1,2,3,4-tetrahydro-6-quinolinecarboxamide

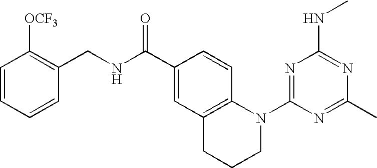

Example 39 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 473.0 [M+H]⁺

### Example 40

### 2-[4-methyl-6-(methylamino)-1,3,5-triazin-2-yl]-N-{[2-(trifluoromethyl)phenyl]methyl}-1,2,3,4-tetrahydro-6-isoquinolinecarboxamide

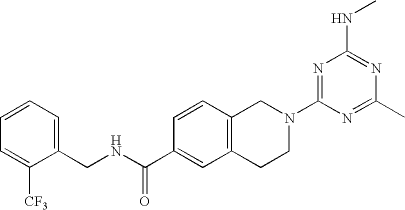

Example 40 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 457.0 [M+H]⁺

### Example 41

### 1-[4-methyl-6-(methylamino)-1,3,5-triazin-2-yl]-N-{[2-(trifluoromethyl)phenyl]methyl}-1,2,3,4-tetrahydro-6-quinolinecarboxamide

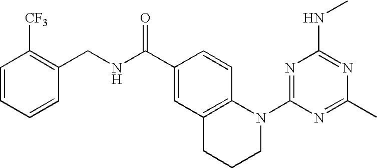

Example 41 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 457.0 [M+H]⁺

### Example 42

### 1-[4-methyl-6-(methylamino)-1,3,5-triazin-2-yl]-N-{[2-(trifluoromethyl)phenyl]methyl}-2,3-dihydro-1H-indole-5-carboxamide

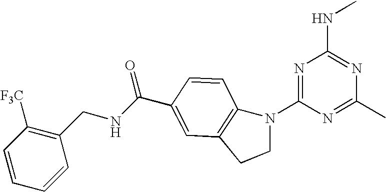

Example 42 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 443.0 [M+H]⁺

### Example 43

### N-[(2,4-dichlorophenyl)methyl]-2-[4-methyl-6-(methylamino)-1,3,5-triazin-2-yl]-1,2,3,4-tetrahydro-6-isoquinolinecarboxamide

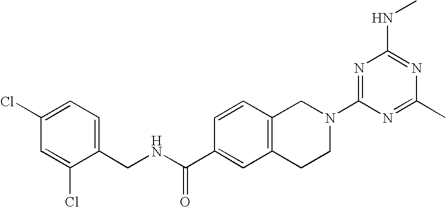

Example 43 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 456.9 [M+H]⁺

### Example 44

### N-[(2,4-dichlorophenyl)methyl]-1-[4-methyl-6-(methylamino)-1,3,5-triazin-2-yl]-1,2,3,4-tetrahydro-6-quinolinecarboxamide

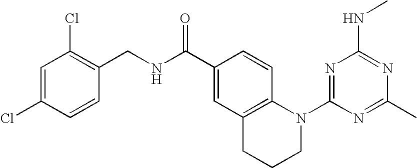

Example 44 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 456.9 [M+H]⁺

### Example 45

### N-[(2,4-dichlorophenyl)methyl]-1-[4-methyl-6-(methylamino)-1,3,5-triazin-2-yl]-2,3-dihydro-1H-indole-5-carboxamide

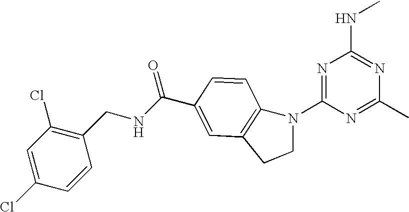

Example 45 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 442.9 [M+H]⁺

### Example 46

### 2-[4-methyl-6-(methylamino)-1,3,5-triazin-2-yl]-N-{[2-(trifluoromethyl)phenyl]methyl}-2,3-dihydro-1H-isoindole-5-carboxamide

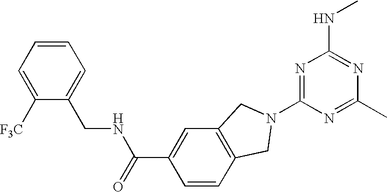

Example 46 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 443.0 [M+H]⁺

### Example 47

### 2-[4-methyl-6-(methylamino)-1,3,5-triazin-2-yl]-N-({2-[(trifluoromethyl)oxy]phenyl}methyl)-2,3-dihydro-1H-isoindole-5-carboxamide

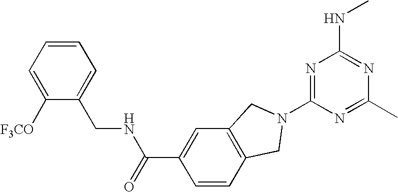

Example 47 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 459.0 [M+H]⁺

### Example 48

### N-[(2,4-dichlorophenyl)methyl]-2-[4-methyl-6-(methylamino)-1,3,5-triazin-2-yl]-2,3-dihydro-1H-isoindole-5-carboxamide

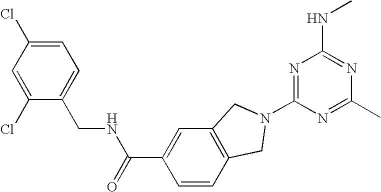

Example 48 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 442.9 [M+H]⁺

### Example 49

### 1-(4-amino-6-phenyl-1,3,5-triazin-2-yl)-N-{[2-(trifluoromethyl)phenyl]methyl}-1,2,3,4-tetrahydro-6-quinolinecarboxamide

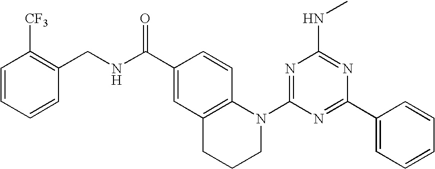

A solution of N-{[2-(trifluoromethyl)phenyl]methyl}-1,2,3,4-tetrahydro-6-quinoline-carboxamide (50.0 mg, 0.15 mmol), prepared similarly to Example 23 and 2,4-dichloro-6-phenyl-1,3,5-triazine (34.0 mg, 0.15 mmol) as prepared in Example 24 in MeCN (3 mL) was heated in the microwave for 15 minutes at 130° C. The reaction was evacuated and transferred to sealed tube. The residue was dissolved in NH₃ (2M in MeOH, 4 mL), sealed, and heated to 60° C. After stirring at 60° C. overnight, the reaction was cooled, evacuated, and purified directly by RP-HPLC (gradient 40-90% CH₃CN:H₂O (0.1% TFA)), to afford the title compound (29 mg, 38%). MS (ES+) m/e 504.9 [M+H]⁺.

### Example 50

### 1-(4-amino-6-phenyl-1,3,5-triazin-2-yl)-N-{[2-(trifluoromethyl)phenyl]methyl}-2,3-dihydro-1H-indole-5-carboxamide

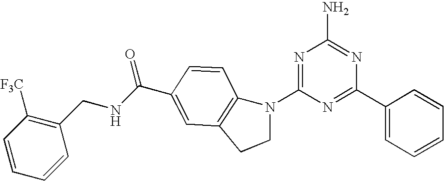

Example 50 was prepared using the general procedure described above in Example 49 substituting the appropriate carboxamide, as prepared using the general procedure described in Example 23, for N-{[2-(trifluoromethyl)phenyl]methyl}-1,2,3,4-tetrahydro-6-quinoline-carboxamide. MS (ES+): m/e 490.9 [M+H]⁺

### Example 51

### N-[(2,4-dichlorophenyl)methyl]-1-[4-(methylamino)-6-phenyl-1,3,5-triazin-2-yl]-1,2,3,4-tetrahydro-5-quinolinecarboxamide

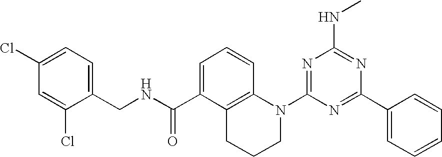

Example 51 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 518.9 [M+H]⁺

### Example 52

### N-[(2,4-dichlorophenyl)methyl]-1-[4-methyl-6-(methylamino)-1,3,5-triazin-2-yl]-1,2,3,4-tetrahydro-5-quinolinecarboxamide

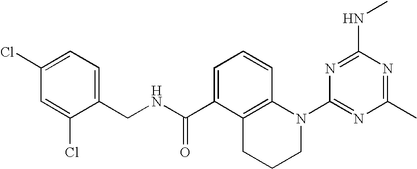

Example 52 was prepared using the general procedure described above in Example 37 substituting the appropriate carboxamide, as prepared using the general procedure described in Example 23, for N-({2-[(trifluoromethyl)oxy]phenyl}methyl)-2,3-dihydro-1H-indole-5-carboxamide. MS (ES+): m/e 456.9 [M+H]⁺

### Example 53

### 1-[4-(methylamino)-6-phenyl-1,3,5-triazin-2-yl]-N-({2-[(trifluoromethyl)oxy]phenyl}methyl)-1,2,3,4-tetrahydro-7-quinolinecarboxamide

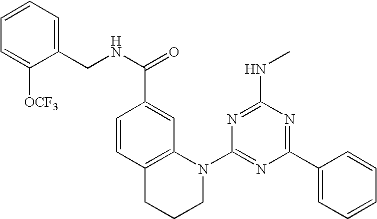

Example 53 was prepared using the general procedure described above in Example 25 substituting the appropriate carboxamide, as prepared using the general procedure described in Example 23, for N-({[2-(trifluoromethyl)oxy]phenyl}methyl)-1,2,3,4-tetrahydro-6-isoquinolinecarboxamide. MS (ES+): m/e 534.9 [M+H]⁺

### Example 54

### 1-[4-(4-chlorophenyl)-6-(methylamino)-1,3,5-triazin-2-yl]-N-[(2,4-dichlorophenyl)methyl]-1,2,3,4-tetrahydro-6-quinolinecarboxamide

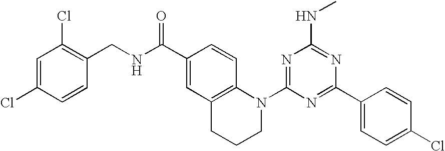

### Step 1: Preparation of 1-[4-chloro-6-(methylamino)-1,3,5-triazin-2-yl]-N-[(2,4-dichlorophenyl)-methyl]-1,2,3,4-tetrahydro-6-quinolinecarboxamide

DIEA (1.39 mL, 8.00 mmol) was added dropwise to a mixture of cyanuric chloride (0.73 g, 3.95 mmol) and N-[(2,4-dichloro-phenyl)methyl]-1,2,3,4-tetrahydro-6-quinolinecarboxamide (1.32 g, 3.95 mmol), prepared similarly to Example 23. After stirring for 45 minutes, an additional 0.2 equivalents of cyanuric chloride were added. This process was repeated until LCMS indicated complete conversion to the desired intermediate. Methylamine (2M THF, 2 mL, 4 mmol) was then added to the reaction. After heating at 50° C. for 30 minutes, LCMS indicated the formation of the desired product. The solvent was removed under vacuum, and triturated with MeOH. The resulting solids were vacuum filtered and washed with MeOH to obtain the title compound (1.45 g, 77%) which was used without any further purification. MS (ES+) m/e 477.0 [M+H]⁺.

### Step 2: 1-[4-(4-chlorophenyl)-6-(methylamino)-1,3,5-triazin-2-yl]-N-[(2,4-dichlorophenyl)methyl]-1,2,3,4-tetrahydro-6-quinolinecarboxamide

To a suspension of 1-[4-chloro-6-(methylamino)-1,3,5-triazin-2-yl]-N-[(2,4-dichlorophenyl)-methyl]-1,2,3,4-tetrahydro-6-quinolinecarboxamide (36 mg, 0.08 mmol), (4-chlorophenyl)boronic acid (14 mg, 0.09 mmol), and Pd(PPh₃)₄ (7 mg, 0.006 mmol) in 1,4-dioxane (3 mL) was added Na₂CO₃ (a.q. (10% W/L)). The reaction mixture was heated in the microwave at 120° C. for 10 minutes and then purified directly by RP-HPLC (gradient; 50-100% CH₃CN:H₂O (0.1% TFA)), to afford the title compound (8.4 mg, 19%). MS (ES+) m/e 553.0 [M+H]⁺.

### Example 55

### N-[(2,4-dichlorophenyl)methyl]-1-[4-(2-fluorophenyl)-6-(methylamino)-1,3,5-triazin-2-yl]-1,2,3,4-tetrahydro-6-quinolinecarboxamide

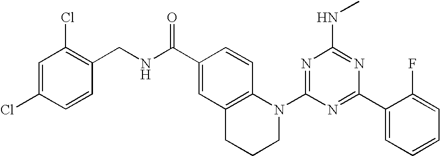

Example 55 was prepared using the general procedure described above in Example 54 substituting the appropriate aryl or heteroaryl boronic acid for 4-chlorophenyl)boronic acid. MS (ES+): m/e 506.8 [M+H]⁺

### Example 56

### N-[(2,4-dichlorophenyl)methyl]-1-{4-(methylamino)-6-[4-(methyloxy)phenyl]-1,3,5-triazin-2-yl}-1,2,3,4-tetrahydro-6-quinolinecarboxamide

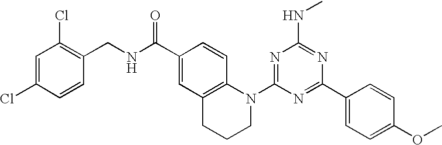

Example 56 was prepared using the general procedure described above in Example 54 substituting the appropriate aryl or heteroaryl boronic acid for 4-chlorophenyl)boronic acid. MS (ES+): m/e 548.9 [M+H]⁺

### Example 57

### N-[(2,4-dichlorophenyl)methyl]-1-[4-(methylamino)-6-(4-pyridinyl)-1,3,5-triazin-2-yl]-1,2,3,4-tetrahydro-6-quinolinecarboxamide

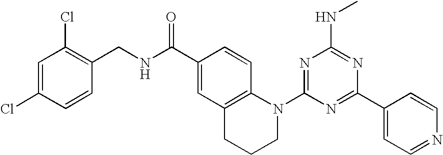

Example 57 was prepared using the general procedure described above in Example 54 substituting the appropriate aryl or heteroaryl boronic acid for 4-chlorophenyl)boronic acid. MS (ES+): m/e 519.9 [M+H]⁺

### Example 58

### N-[(2,4-dichlorophenyl)methyl]-1-[4-(2,4-difluorophenyl)-6-(methylamino)-1,3,5-triazin-2-yl]-1,2,3,4-tetrahydro-6-quinolinecarboxamide

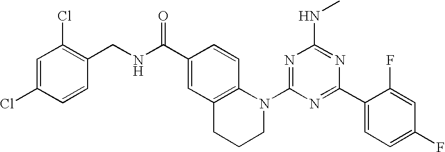

Example 58 was prepared using the general procedure described above in Example 54 substituting the appropriate aryl or heteroaryl boronic acid for 4-chlorophenyl)boronic acid. MS (ES+): m/e 554.9 [M+H]⁺

### Example 59

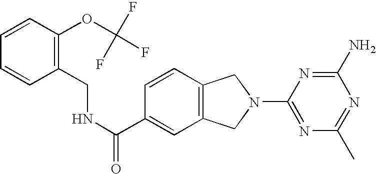

### 2-(4-amino-6-methyl-1,3,5-triazin-2-yl)-N-({2-[(trifluoromethyl)oxy]phenyl}methyl)-2,3-dihydro-1H-isoindole-5-carboxamide

2-(4-amino-6-methyl-1,3,5-triazin-2-yl)-2,3-dihydro-1H-isoindole-5-carboxylic acid (45 mg, 0.166 mmol), 1H-1,2,3-benzotriazol-1-yloxy-tris(dimethylamino)-phosphonium hexafluorophosphate (BOP reagent, 88 mg, 0.199 mmol), 2-trifluoromethoxybenzylamine (32 mg, 0.166 mmol) and diisopropylethylamine (43 mg, 0.332 mmol) were combined in 3 mL of DMF and stirred at room temperature for 3 hours. The reaction mixture was purified by preparative HPLC to provide 2-(4-amino-6-methyl-1,3,5-triazin-2-yl)-N-({2-[(trifluoromethyl)oxy]phenyl}methyl)-2,3-dihydro-1H-isoindole-5-carboxamide (65 mg, 0.116 mmol, 70%) as the TFA salt. MS (ES) m/e 446 [M+H]⁺. ¹H NMR (400 MHz, Methanol-D4) □7.9 (m, 2H), 7.5 (m, 2H), 7.4 (m, 3H), 5.1 (s, 2H), 5.0 (s, 2H), 4.7 (s, 2H), 2.5 (s, 3H)

### Example 60

### 2-(4-amino-6-methyl-1,3,5-triazin-2-yl)-N-({4-(1-pyrrolidinyl)-2-[(trifluoromethyl)oxy]phenyl}methyl)-2,3-dihydro-1H-isoindole-5-carboxamide

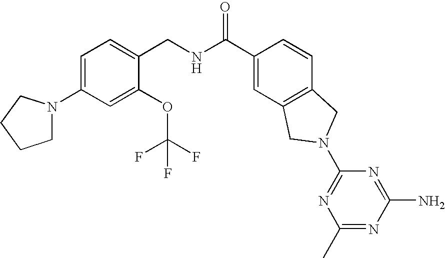

### a) 2-(4-amino-6-methyl-1,3,5-triazin-2-yl)-N-({4-bromo-2-[(trifluoromethyl)oxy]phenyl}methyl)-2,3-dihydro-1H-isoindole-5-carboxamide

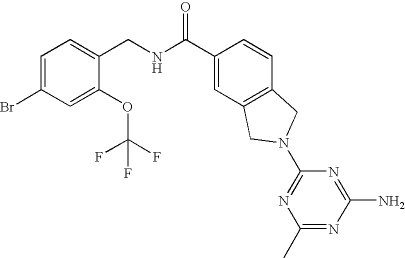

2-(4-amino-6-methyl-1,3,5-triazin-2-yl)-N-({4-bromo-2-[(trifluoromethyl)oxy]phenyl}methyl)-2,3-dihydro-1H-isoindole-5-carboxamide was prepared using the general procedure described above in Example 59 substituting 1-{4-bromo-2-[(trifluoromethyl)oxy]phenyl}methanamine for 2-trifluoromethoxybenzylamine. MS (ES+): m/e 525 [M+H]⁺

### b) 2-(4-amino-6-methyl-1,3,5-triazin-2-yl)-N-({4-(1-pyrrolidinyl)-2-[(trifluoromethyl)oxy]phenyl}methyl)-2,3-dihydro-1H-isoindole-5-carboxamide

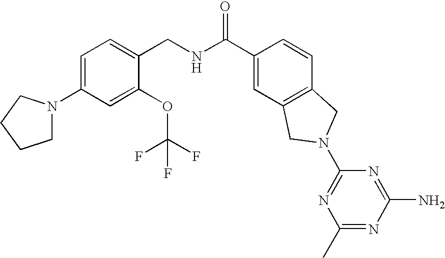

A mixture of 2-(4-amino-6-methyl-1,3,5-triazin-2-yl)-N-({4-bromo-2-[(trifluoromethyl)oxy]phenyl}methyl)-2,3-dihydro-1H-isoindole-5-carboxamide (50 mg, 0.096 mmol), pyrrolidine, Pd₂(dba)₃ (4.4 mg, 4 μmol), BINAP (4.5 mg, 7 μmol) and cesium carbonate (44 mg, 0.134 mmol) was combined in 1,4-dioxane (10 mL) and heated to 95° C. and maintained at that temperature overnight. The reaction mixture was filtered through Celite, concentrated to dryness, and purified by preparative HPLC to provide 2-(4-amino-6-methyl-1,3,5-triazin-2-yl)-N-({4-(1-pyrrolidinyl)-2-[(trifluoromethyl)oxy]phenyl}methyl)-2,3-dihydro-1H-isoindole-5-carboxamide (4.5 mg, 7 □mol, 8%) as the TFA salt. MS (ES+): m/e 514 [M+H]⁺

### Example 61

### 2-(4-amino-6-methyl-1,3,5-triazin-2-yl)-N-{[2-chloro-4-(dimethylamino)phenyl]methyl}-2,3-dihydro-1H-isoindole-5-carboxamide

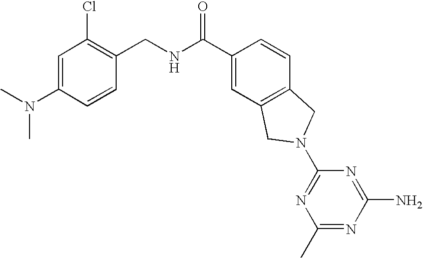

Example 61 was prepared using the general procedure described above in Example 59 substituting [4-(aminomethyl)-3-chlorophenyl]dimethylamine for 2-trifluoromethoxybenzylamine. MS (ES+): m/e 438 [M+H]⁺

### Example 62

### 2-(4-amino-6-methyl-1,3,5-triazin-2-yl)-N-{[4-chloro-2-(trifluoromethyl)phenyl]methyl}-2,3-dihydro-1H-isoindole-5-carboxamide

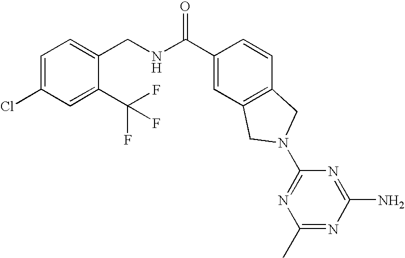

Example 62 was prepared using the general procedure described above in Example 59 substituting {[4-chloro-2-(trifluoromethyl)phenyl]methyl}amine for 2-trifluoromethoxybenzylamine. MS (ES+): m/e 463 [M+H]⁺

### Example 63

### N-({2-chloro-4-[(methylsulfonyl)amino]phenyl}methyl)-1-[4-methyl-6-(methylamino)-1,3,5-triazin-2-yl]-2,3-dihydro-1H-indole-5-carboxamide

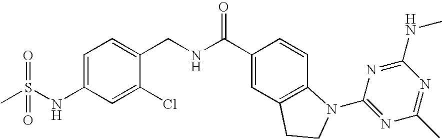

Example 63 was prepared using the general procedure described above in Example 59 substituting 1-[4-methyl-6-(methylamino)-1,3,5-triazin-2-yl]-2,3-dihydro-1H-indole-5-carboxylic acid for 2-(4-amino-6-methyl-1,3,5-triazin-2-yl)-2,3-dihydro-1H-isoindole-5-carboxylic acid and substituting N-[4-(aminomethyl)-3-chlorophenyl]methanesulfonamide for trifluoromethoxybenzylamine. MS (ES+): m/e 502 [M+H]⁺

### Example 64

### 1-(4-amino-6-methyl-1,3,5-triazin-2-yl)-N-({2-[(trifluoromethyl)oxy]phenyl}methyl)-2,3-dihydro-1H-indole-5-carboxamide

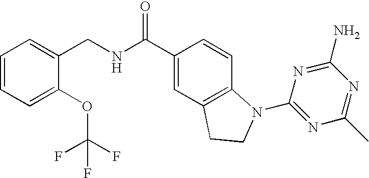

### a) 1-(4-chloro-6-methyl-1,3,5-triazin-2-yl)-N-({2-[(trifluoromethyl)oxy]phenyl}methyl)-2,3-dihydro-1H-indole-5-carboxamide

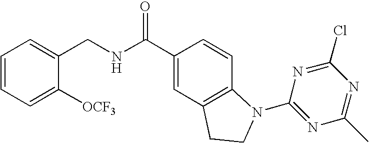

N-({2-[(trifluoromethyl)oxy]phenyl}methyl)-2,3-dihydro-1H-indole-5-carboxamide (1.76 g, 5.23 mmol), 2,4-dichloro-6-methyl-1,3,5-triazine (0.858 g, 5.23 mmol), and diisopropylethylamine (1.83 mL, 10.5 mmol) were dissolved in 30 mL of acetonitrile and stirred for one hour. The solvent was removed under vacuum to afford crude 1-(4-chloro-6-methyl-1,3,5-triazin-2-yl)-N-({2-[(trifluoromethyl)oxy]phenyl}methyl)-2,3-dihydro-1H-indole-5-carboxamide (2.2 g, 4.74 mmol, 91%) which was used in the next step without further purification. MS (ES+): m/e 464, 466 [M+H]⁺

### b) 1-(4-amino-6-methyl-1,3,5-triazin-2-yl)-N-({2-[(trifluoromethyl)oxy]phenyl}methyl)-2,3-dihydro-1H-indole-5-carboxamide

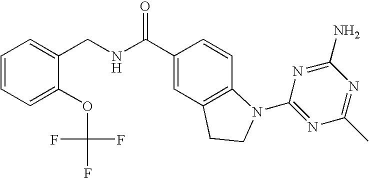

1-(4-chloro-6-methyl-1,3,5-triazin-2-yl)-N-({2-[(trifluoromethyl)oxy]phenyl}methyl)-2,3-dihydro-1H-indole-5-carboxamide (100 mg, 0.216 mmol) was dissolved in 3 mL of acetonitrile and treated with excess aqueous ammonium hydroxide. The reaction was heated to 50° C. in a sealed tube for 2 hours. The reaction mixture was purified directly by preparative HPLC to provide 1-(4-amino-6-methyl-1,3,5-triazin-2-yl)-N-({2-[(trifluoromethyl)oxy]phenyl}methyl)-2,3-dihydro-1H-indole-5-carboxamide (23 mg, 0.039 mmol, 18%) as the TFA salt. MS (ES+): m/e 445 [M+H]⁺

As used above, the phrase “using the general procedure described above” indicates that the procedure used employs similar, but not necessarily identical, reaction conditions to those referred to.

### Biological Activity

The compounds according to Formula I are sEH inhibitors. The compounds according to Formula I, therefore, are useful in the treatment of hypertension and other conditions involving sEH activity. As stated above, mEH provides an important detoxification pathway in mammals. Compounds that exhibit pharmacological selectivity for sEH over mEH therefore are desirable in the methods of treatment described below. Accordingly, in one embodiment the invention is directed to a compound according to Formula I wherein the compound exhibits a selectivity ratio equal to or greater than 10:1 for sEH over mEH. In another embodiment the invention is directed to a compound according to Formula I wherein the compound exhibits a selectivity ratio equal to or greater than 100:1 for sEH over mEH. In another embodiment the invention is directed to a compound according to Formula I wherein the compound exhibits a selectivity ratio equal to or greater than 1000:1 for sEH over mEH.

The biological activity of the compounds according to Formula I can be determined using any suitable assay for determining the activity of a candidate compound as an sEH and/or mEH inhibitor, as well as suitable tissue and/or in vivo models.

### In Vitro Fluorescence Assay

Inhibition of Soluble Expoxide Hydrolase (sEH) activity is measured in a fluorescent assay based upon the format described by Wolf et al. (Analytical Biochemistry Vol. 355 (2006) pp. 71-80). In the presence of sEH, PHOME ((3-Phenyl-oxiranyl)-acetic acid cyano-(6-methoxy-naphthalen-2-yl)-methyl ester), is hydrolyzed to a diol which goes through an intramolecular cyclization and the release and decomposition of cyanohydrin (products=cyanide and 6-methoxy-2-naphthaldehyde). Production of 6-methoxy-2-naphthaldehyde is monitored at excitation of 360 nm and an emission of 465 nm.

The assay is used in a quenched assay format by sequentially adding enzyme (5 uL; 200 pM sEH in 25 mM Hepes at pH 7.0, 0.01% CHAPS (w/v), 0.005% Casein (w/v); 10 minute ambient pre-incubation after addition) then PHOME substrate (5 ul; 10 uM PHOME substrate in 25 mM Hepes at pH 7.0, 0.01% CHAPS (w/v), 0.005% Casein (w/v)) to a 384 well assay plate (Greiner 784076) pre-stamped with 25-100 nL compound at the desired concentration. The reaction is incubated for 30 minutes at room temperature, then quenched by the addition of stop solution (5 uL; 10 mM ZnSO4 in 25 mM Hepes at pH 7.0, 0.01% CHAPS (w/v), 0.005% Casein (w/v)). Microtiter plates are centrifuged after each addition for 30 seconds at 500 rpm. The fluorescence is measured on an EnVision plate reader platform (Perkin Elmer) using a 360 nm excitation filter, 465 nm emission filter, and 400 nm dichroic filter.

Compounds are first prepared in neat DMSO at a concentration of 10 mM, then diluted as required to achieve the desired assay concentration. For inhibition curves, compounds are diluted using a three fold serial dilution and tested at 11 concentrations (e.g. 50 μM-0.8 nM or 25 μM-0.42 nM or 2.5 μM to 42 pM). Curves are analysed using ActivityBase and XLfit, and results are expressed as pIC50 values.

### Cell-Based sEH Inhibitor Assay

Cell based sEH inhibition is measured using the 14,15-DHET immunoassay ELISA kit available from Detroit R&D (Cat. No. DH1), according to the following procedure:

HEK293 cells (BioCat ID 80556) are transduced by sEH BacMam virus to increase sEH expression (other cell lines may be suitahle) as follows: One day before the experiment, 1.5 million HEK293 cells (BioCat ID 80556) are seated in 3 ml of DMEM/F12 (with L-Glutamine, with 15 mM HEPES, pH7.30, from Media Prep Lab), with 10% fetal bovine serum (from SAFC Biosciences, Cat. No. 12176-1000M), no antibiotic, in a 25 cm² flask (from Corning Incorporated, Cat. No. 430639) and 30 μL sEH BacMam virus is added. The cells are gently mixed then incubated at 37° C., 5% CO₂, for 24 hours.

The cells are trypsinized to release them from the growth flask, washed once with PBS, then re-suspended in 5 mL DMEM/F12 without phenol red (from Media Prep lab). Cell density should be approximately 3*10⁵ cells/mL (=300 cells/μL), counted using the Cedex AS²⁰ (from Innovatis).

The cells are then diluted in DMEM/F12 to 5.1 cells/μL, and 98 μL/well (=500 cells/well) of this cell suspension is transferred to an assay plate (96 well, clear polystyrene, flat bottom, from Whatman, Cat. No. 7701-1350).

2 μL of the diluted test compound is then added to the cells in the assay plate. The reaction plate is shaken gently and incubated at room temperature for 30 min, after which 10 μL of substrate solution is added (substrate solution is prepared by diluting 1.24 μL of 14,15-EET from Cayman Chemical, Cat. No. 50651 with 8.24 μL DMEM/F12). The assay plate is then incubated for one hour at room temperature.

After the 1 hour reaction, the reaction mixture is diluted 3 fold with provided sample dilution buffer (ex. Add 220 μL to the 110 μL reaction mixture), mixed well, and spun for 5 min at 500 rpm.

100 μL of the diluted reaction mixture is then transferred from the reaction plates to the ELISA plates, and the ELISA is performed according to the instructions provided in the kit.

IC50s and pIC50s are then calculated. The IC50 can be calculated directly using the 14, 15-DHET concentration or using the % inhibition [% inhibition=100*(1−(sample DHET−0 cell DHET)/(500 cells DHET−0 cell DHET)].

Compounds are first prepared in neat DMSO at a concentration of 0.5 mM, then diluted as required to achieve the desired assay concentration. For inhibition curves, compounds are diluted using a three fold serial dilution and tested at 9 concentrations (e.g. 10 μM-1.5 nM). Curves are analysed using ActivityBase and XLfit, and results are expressed as pIC50 values.

### Biological Activity Results

All of the compounds exemplified above were tested for activity as sEH inhibitors. Where the assay for a particular compound had been performed two or more times, the following conclusion regarding their activities is based on the average of individual experiments. All exemplified compounds were found to have an IC50 in the range of 0.1 and 10,000 nM.

### Methods of Use

The compounds of the invention inhibit the sEH enzyme and can be useful in the treatment of conditions wherein the underlying pathology is (at least in part) attributable to sEH involvement or in conditions wherein sEH inhibition offers some clinical benefit even though the underlying pathology is not (even in part) attributable to sEH involvement. Examples of such conditions include hypertension, organ failure/damage (including heart failure, cardiac and renal fibrosis, renal failure, and liver failure), peripheral vascular disease (including ischemic limb disease, intermittent claudication, endothelial dysfunction, erectile dysfunction, Raynaud's disease, and diabetic vasculopathies e.g. retinopathy), atherothrombotic disorders (including coronary artery disease, coronary vasospasm, angina, stroke, myocardial ischemia, myocardial infarction, and hyperlipidemia), metabolic disorders (including diabetes), and inflammatory disorders (including arthritis, inflammatory pain, overactive bladder, asthma, and COPD). Accordingly, in another aspect the invention is directed to methods of treating such conditions.

Essential hypertension is commonly associated with the development of significant end organ damage such as renal, endothelial, myocardial, and erectile dysfunction. Such conditions occur “secondary” to the elevated systemic arterial blood pressure. Secondary conditions may be prevented by treatment of the underlying (“primary”) cause. Accordingly, in another aspect the invention is directed to methods of preventing such secondary conditions.

Heart failure is a complex heterogenous disorder characterized by reduced cardiac output, resulting in the inability of the heart to meet perfusion demands of the body. Cardiac proinflammatory cytokine recruitment and maladaptive cardiac hypertrophy, fibrosis and apoptosis/necrosis are factors associated with the progression of heart failure. Compounds of the invention are directed to methods of treating such conditions.

In addition, sEH is indirectly involved in the regulation of platelet function through its effect on EETs. Drugs that inhibit platelet aggregation are believed to decrease the risk of atherthrombotic events, such as myocardial infarction and stroke, in patients with established cardiovascular atherosclerotic disease. Accordingly, in another aspect the invention is directed to methods of preventing atherothrombotic events, such as myocardial infarction and stroke in patients with a history of recent myocardial infarction, stroke, transient ischemic attacks, unstable angina, or atherosclerosis.

The methods of treating and the methods of preventing described above comprise administering a safe and effective amount of a compound of the invention to a patient in need thereof.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of the invention or other pharmaceutically-active agent means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient being treated; the duration of the treatment; the nature of concurrent therapy; the desired therapeutic effect; and like factors, but can nevertheless be determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders, syrups, and solutions for injection. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excepient or excepients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

1. A compound according to Formula I: 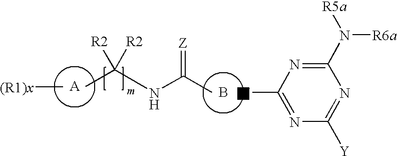
Formula I
wherein:
A is phenyl, monocyclic heteroaryl, or C5-C6 cycloalkyl;
when A is phenyl or monocyclic heteroaryl each R1 is independently selected from the group consisting of: halo, CN, Ra, ORb, C(O)ORc, C(O)NRcRc, NRcRc, NRcC(O)Rb, NRcS(O₂)Ra, SRb, S(O₂)Ra, and S(O₂)NRcRc;
when A is C5-C6 cycloalkyl each R1 is independently selected from the group consisting of: Ra, ORb, C(O)ORc, C(O)NRcRc, NRcRc, and NRcC(O)Rb;
x is an integer from 0 to 5;
each R2 is independently H or C1-C3 alkyl;
m is 1 or 2;
Z is O or S;
B is B1, B2, B3, B4, B5, B6, B7, B8, B9, B10, B11, or B12;
B1 is 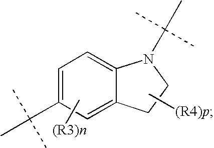
B2 is 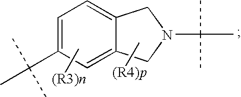
B3 is 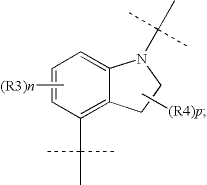
B4 is 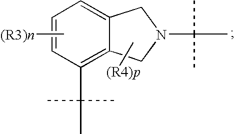
B5 is 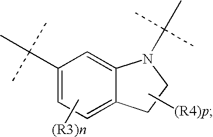
B6 is 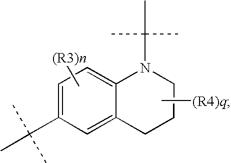
B7 is 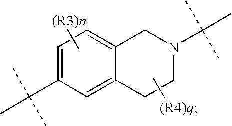
B8 is 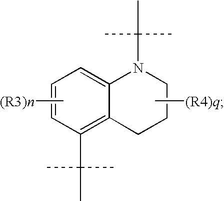
B9 is 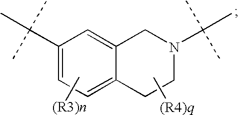
B10 is 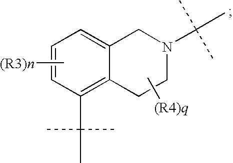
B11 is 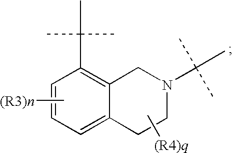
B12 is 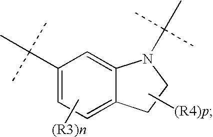
R3, if present, is a substituent on the phenyl ring of said B ring system and each R3 is independently selected from the group consisting of: halo and C1-C3 alkyl;
n is an integer from 0 to 3;
R4, if present, is a substituent on the Nitrogen-containing ring of said B ring system and each R4 is independently C1-C3 alkyl;
p is an integer from 0 to 2;
q is an integer from 0 to 4;
Y is H, OH, R7, R8, R9, R10, R11, R12, or NR5bR6b;
R5a and R5b are each independently H, R51, R52, R53, R54, R55, —C(O)Rb, —C(O)NRcRc, —S(O₂)Ra, or —S(O₂)NRcRc;
each R51 is C1-C6 alkyl optionally substituted with one or more substituents selected from the group consisting of: halo, ORd, SRk, C(O)ORc, C(O)NReRe, NReRe, Rg, Rh, Ri, Rj;
each R52 is C3-C6 cycloalkyl optionally substituted with one or more substituents selected from the group consisting of: halo, ORd, SRd, C(O)ORc, C(O)NReRe, NReRe, C1-C3 alkyl, and C1-C3 haloalkyl;
R53 is monocyclic heterocycloalkyl optionally substituted with one or more C1-C3 alkyl;
R54 is phenyl optionally substituted with one or more substituents selected from the group consisting of: halo, CN, Ra, ORb, C(O)ORc, C(O)NRcRc, NRcRc, NRcC(O)Rb, NRcS(O₂)Ra, SRb, S(O₂)Ra, and S(O₂)NReRe;
R55 is monocyclic heteroaryl optionally substituted with one or more substituents selected from the group consisting of: halo, —CN, C1-C3 alkyl, C1-C3 haloalkyl, ORd, and NReRe;
R6a and R6b are each independently H, R51, or R52; or
R5a and R6a and/or R5b and R6b, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;
R7 is C1-C8 alkyl optionally substituted with one or more substituents selected from the group consisting of: halo, ORd, SRd, NReRe, C3-C6 cycloalkyl, Ri, and Rj;
R8 is C3-C6 cycloalkyl optionally substituted with one or more substituents selected from the group consisting of: halo, ORd, SRd, NReRe, C1-C3 alkyl, and C1-C3 haloalkyl;
R9 monocyclic heterocycloalkyl optionally substituted with one or more C1-C3 alkyl;
R10 is phenyl optionally substituted with one or more substituents selected from the group consisting of: halo, CN, Ra, ORb, C(O)ORc, C(O)NReRe, NReRe, NRcC(O)Rb, NRcS(O₂)Ra, SRb, S(O₂)Ra, and S(O₂)NRcRc
R11 is heteroaryl optionally substituted with one or more substituents selected from the group consisting of: halo, CN, Ra, ORb, C(O)ORc, C(O)NReRe, NReRe, NRcC(O)Rb, NRcS(O₂)Ra, SRb, S(O₂)Ra, and S(O₂)NRcRc;
R12 is —OR7, —OR8, —OR9, —OR10, —OR11, —SR7, —SR8, —SR9, —SR10, or SR11;
each Ra is independently C1-C6 alkyl or C1-C6 haloalkyl;
each Rb is independently H, C1-C6 alkyl or C1-C6 haloalkyl;
each Rc is independently H or C1-C6 alkyl;
where there are two Rc groups attached to a nitrogen;
both Rc groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;
each Rd is independently H, C1-C3 alkyl or C1-C3 haloalkyl;
each Re is independently H, C1-C3 alkyl, CH₂—CF₃; or
both Re groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;
each Rf is independently H or C1-C3 alkyl.
each Rg is C3-C6 cycloalkyl optionally substituted with one or more substituents selected from the group consisting of: halo, ORd, SRd, C(O)ORc, C(O)NReRe, NReRe, and C1-C3 alkyl;
each Rh is monocyclic heterocycloalkyl optionally substituted with one or more C1-C3 alkyl;
each Ri is phenyl optionally substituted with one or more substituents selected from the group consisting of: halo, —CN, C1-C3 alkyl, C1-C3 haloalkyl, ORd, and NReRe;
each Rj is monocyclic heteroaryl optionally substituted with one or more substituents selected from the group consisting of: halo, —CN, C1-C3 alkyl, C1-C3 haloalkyl, ORd, and NReRe; and
each Rk is independently H, C1-C3 alkyl, C1-C3 haloalkyl, or benzyl optionally substituted with one or more substituents selected from the group consisting of: halo, —CN, C1-C3 alkyl, C1-C3 haloalkyl, ORd, and NReRe;
or a pharmaceutically acceptable salt thereof.

2. A compound of claim 1 wherein:
A is phenyl, thiophenyl, or pyridyl;
R1 is CF₃, halo, OCF₃, CN, OC1-C6 alkyl, morpholino, CO₂H, or N(CH₃)₂;
x is 1, 2, or 3;
B is B1, B2, B6, and B7;
n is 0;
Z is O;
Y is C1-C3 alkyl, phenyl, thiophenyl, or pyridyl; wherein the phenyl, thiophenyl or pyridyl may be substituted by —CO₂H, SO₂Me, CF₃, halo, or CN;
R5a is hydrogen or C1-C6 alkyl; and
R6a is hydrogen or C1-C6 alkyl;
or a pharmaceutically acceptable salt thereof.

3. A compound of claim 1 wherein:
A is phenyl;
R1 is CF₃, halo, OCF₃, CN, OC1-C6 alkyl, or morpholino;
x is 1, or 2;
B is;
n is 0
Z is O;
Y is methyl;
R5a is hydrogen; and
R6a is;
or a pharmaceutically acceptable salt thereof.

4. A compound of claim 1 chosen from:
Specific examples of compounds of the present invention include the following:
N-[(2,4-dichlorophenyl)methyl]-2-[4-(methylamino)-6-(4-methyl-1-piperazinyl)-1,3,5-triazin-2-yl]-2,3-dihydro-1H-isoindole-5-carboxamide;
2-[4-(methylamino)-6-(4-methyl-1-piperazinyl)-1,3,5-triazin-2-yl]-N-{[2-(trifluoromethyl)phenyl]methyl}-2,3-dihydro-1H-isoindole-5-carboxamide trifluoroacetate;
2-[4-(methylamino)-6-(4-methyl-1-piperazinyl)-1,3,5-triazin-2-yl]-N-({2-[(trifluoromethyl)oxy]phenyl}methyl)-2,3-dihydro-1H-isoindole-5-carboxamide trifluoroacetate;
N-{[4-chloro-2-(trifluoromethyl)phenyl]methyl}-2-[4-(methylamino)-6-(4-methyl-1-piperazinyl)-1,3,5-triazin-2-yl]-2,3-dihydro-1H-isoindole-5-carboxamide trifluoroacetate;
2-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-isoquinolinecarboxamide;
2-[4-(methylamino)-6-(4-methyl-1-piperazinyl)-1,3,5-triazin-2-yl]-N-{[2-(trifluoromethyl)phenyl]methyl}-1,2,3,4-tetrahydro-6-isoquinolinecarboxamide;
2-[4-(methylamino)-6-(4-methyl-1-piperazinyl)-1,3,5-triazin-2-yl]-N-{[4-(trifluoromethyl)phenyl]methyl}-1,2,3,4-tetrahydro-6-isoquinolinecarboxamide;
N-[(2,5-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide;
4-{[({2-[4-(methylamino)-6-(4-methyl-1-piperazinyl)-1,3,5-triazin-2-yl]-1,2,3,4-tetrahydro-6-isoquinolinyl}carbonyl)amino]methyl}benzoic acid;
N-[(2,4-dichlorophenyl)methyl]-2-[4-(methylamino)-6-(4-methyl-1-piperazinyl)-1,3,5-triazin-2-yl]-1,2,3,4-tetrahydro-6-isoquinolinecarboxamide;
N-{[4-chloro-2-(trifluoromethyl)phenyl]methyl}-2-[4-(methylamino)-6-(4-methyl-1-piperazinyl)-1,3,5-triazin-2-yl]-1,2,3,4-tetrahydro-6-isoquinolinecarboxamide;
1-[4-(methylamino)-6-(4-methyl-1-piperazinyl)-1,3,5-triazin-2-yl]-N-{[2-(trifluoromethyl)phenyl]methyl}-1,2,3,4-tetrahydro-6-quinolinecarboxamide;
1-[4-(methylamino)-6-(4-methyl-1-piperazinyl)-1,3,5-triazin-2-yl]-N-({2-[(trifluoromethyl)oxy]phenyl}methyl)-1,2,3,4-tetrahydro-6-quinolinecarboxamide;
N-[(2,4-dichlorophenyl)methyl]-1-[4-(methylamino)-6-(4-methyl-1-piperazinyl)-1,3,5-triazin-2-yl]-1,2,3,4-tetrahydro-6-quinolinecarboxamide;
N-[(2,5-dichlorophenyl)methyl]-1-[4-(methylamino)-6-(4-methyl-1-piperazinyl)-1,3,5-triazin-2-yl]-1,2,3,4-tetrahydro-6-quinolinecarboxamide;
1-[4-(methylamino)-6-(4-methyl-1-piperazinyl)-1,3,5-triazin-2-yl]-N-{[4-(trifluoromethyl)phenyl]methyl}-1,2,3,4-tetrahydro-6-quinolinecarboxamide;
1-[4-(methylamino)-6-(4-methyl-1-piperazinyl)-1,3,5-triazin-2-yl]-N-{[2-(methyloxy)phenyl]methyl}-1,2,3,4-tetrahydro-6-quinolinecarboxamide;
N-[(3,4-difluorophenyl)methyl]-1-[4-(methylamino)-6-(4-methyl-1-piperazinyl)-1,3,5-triazin-2-yl]-1,2,3,4-tetrahydro-6-quinolinecarboxamide;
1-[4-(methylamino)-6-(4-methyl-1-piperazinyl)-1,3,5-triazin-2-yl]-N-{[2-(trifluoromethyl)phenyl]methyl}-2,3-dihydro-1H-indole-5-carboxamide;
1-[4-(methylamino)-6-(4-methyl-1-piperazinyl)-1,3,5-triazin-2-yl]-N-({2-[(trifluoromethyl)oxy]phenyl}methyl)-2,3-dihydro-1H-indole-5-carboxamide trifluoroacetate;
N-[(2,4-dichlorophenyl)methyl]-1-[4-(methylamino)-6-(4-methyl-1-piperazinyl)-1,3,5-triazin-2-yl]-2,3-dihydro-1H-indole-5-carboxamide trifluoroacetate;
N-{[4-chloro-2-(trifluoromethyl)phenyl]methyl}-1-[4-(methylamino)-6-(4-methyl-1-piperazinyl)-1,3,5-triazin-2-yl]-2,3-dihydro-1H-indole-5-carboxamide trifluoroacetate;
N-({2-[(trifluoromethyl)oxy]phenyl}methyl)-1,2,3,4-tetrahydro-6-isoquinolinecarboxamide 2,4-dichloro-6-phenyl-1,3,5-triazine;
2-[4-(methylamino)-6-phenyl-1,3,5-triazin-2-yl]-N-({2-[(trifluoromethyl)oxy]phenyl}methyl)-1,2,3,4-tetrahydro-6-isoquinolinecarboxamide;
1-[4-(methylamino)-6-phenyl-1,3,5-triazin-2-yl]-N-{[2-(trifluoromethyl)phenyl]methyl}-1,2,3,4-tetrahydro-6-quinolinecarboxamide;
1-[4-(methylamino)-6-phenyl-1,3,5-triazin-2-yl]-N-{[2-(trifluoromethyl)phenyl]methyl}-2,3-dihydro-1H-indole-5-carboxamide;
2-[4-(methylamino)-6-phenyl-1,3,5-triazin-2-yl]-N-{[2-(trifluoromethyl)phenyl]methyl}-1,2,3,4-tetrahydro-6-isoquinolinecarboxamide;
N-[(2,4-dichlorophenyl)methyl]-1-[4-(methylamino)-6-phenyl-1,3,5-triazin-2-yl]-2,3-dihydro-1H-indole-5-carboxamide;
N-[(2,4-dichlorophenyl)methyl]-1-[4-(methylamino)-6-phenyl-1,3,5-triazin-2-yl]-1,2,3,4-tetrahydro-6-quinolinecarboxamide;
1-[4-(methylamino)-6-phenyl-1,3,5-triazin-2-yl]-N-({2-[(trifluoromethyl)oxy]phenyl}methyl)-2,3-dihydro-1H-indole-5-carboxamide;
1-[4-(methylamino)-6-phenyl-1,3,5-triazin-2-yl]-N-({2-[(trifluoromethyl)oxy]phenyl}methyl)-1,2,3,4-tetrahydro-6-quinolinecarboxamide;
N-[(2,4-dichlorophenyl)methyl]-2-[4-(methylamino)-6-phenyl-1,3,5-triazin-2-yl]-2,3-dihydro-1H-isoindole-5-carboxamide;
2-[4-(methylamino)-6-phenyl-1,3,5-triazin-2-yl]-N-{[2-(trifluoromethyl)phenyl]methyl}-2,3-dihydro-1H-isoindole-5-carboxamide;
2-[4-(methylamino)-6-phenyl-1,3,5-triazin-2-yl]-N-({2-[(trifluoromethyl)oxy]phenyl}methyl)-2,3-dihydro-1H-isoindole-5-carboxamide;
N-[(2,4-dichlorophenyl)methyl]-2-[4-(methylamino)-6-phenyl-1,3,5-triazin-2-yl]-1,2,3,4-tetrahydro-6-isoquinolinecarboxamide trifluoroacetate;
1-[4-methyl-6-(methylamino)-1,3,5-triazin-2-yl]-N-({2-[(trifluoromethyl)oxy]phenyl}methyl)-2,3-dihydro-1H-indole-5-carboxamide;
2-[4-methyl-6-(methylamino)-1,3,5-triazin-2-yl]-N-({2-[(trifluoromethyl)oxy]phenyl}methyl)-1,2,3,4-tetrahydro-6-isoquinolinecarboxamide;
1-[4-methyl-6-(methylamino)-1,3,5-triazin-2-yl]-N-({2-[(trifluoromethyl)oxy]phenyl}methyl)-1,2,3,4-tetrahydro-6-quinolinecarboxamide;
2-[4-methyl-6-(methylamino)-1,3,5-triazin-2-yl]-N-{[2-(trifluoromethyl)phenyl]methyl}-1,2,3,4-tetrahydro-6-isoquinolinecarboxamide;
1-[4-methyl-6-(methylamino)-1,3,5-triazin-2-yl]-N-{[2-(trifluoromethyl)phenyl]methyl}-1,2,3,4-tetrahydro-6-quinolinecarboxamide;
1-[4-methyl-6-(methylamino)-1,3,5-triazin-2-yl]-N-{[2-(trifluoromethyl)phenyl]methyl}-2,3-dihydro-1H-indole-5-carboxamide;
N-[(2,4-dichlorophenyl)methyl]-2-[4-methyl-6-(methylamino)-1,3,5-triazin-2-yl]-1,2,3,4-tetrahydro-6-isoquinolinecarboxamide;
N-[(2,4-dichlorophenyl)methyl]-1-[4-methyl-6-(methylamino)-1,3,5-triazin-2-yl]-1,2,3,4-tetrahydro-6-quinolinecarboxamide;
N-[(2,4-dichlorophenyl)methyl]-1-[4-methyl-6-(methylamino)-1,3,5-triazin-2-yl]-2,3-dihydro-1H-indole-5-carboxamide;
2-[4-methyl-6-(methylamino)-1,3,5-triazin-2-yl]-N-{[2-(trifluoromethyl)phenyl]methyl}-2,3-dihydro-1H-isoindole-5-carboxamide;
2-[4-methyl-6-(methylamino)-1,3,5-triazin-2-yl]-N-({2-[(trifluoromethyl)oxy]phenyl}methyl)-2,3-dihydro-1H-isoindole-5-carboxamide;
N-[(2,4-dichlorophenyl)methyl]-2-[4-methyl-6-(methylamino)-1,3,5-triazin-2-yl]-2,3-dihydro-1H-isoindole-5-carboxamide;
1-(4-amino-6-phenyl-1,3,5-triazin-2-yl)-N-{[2-(trifluoromethyl)phenyl]methyl}-1,2,3,4-tetrahydro-6-quinolinecarboxamide;
1-(4-amino-6-phenyl-1,3,5-triazin-2-yl)-N-{[2-(trifluoromethyl)phenyl]methyl}-2,3-dihydro-1H-indole-5-carboxamide;
N-[(2,4-dichlorophenyl)methyl]-1-[4-(methylamino)-6-phenyl-1,3,5-triazin-2-yl]-1,2,3,4-tetrahydro-5-quinolinecarboxamide;
N-[(2,4-dichlorophenyl)methyl]-1-[4-methyl-6-(methylamino)-1,3,5-triazin-2-yl]-1,2,3,4-tetrahydro-5-quinolinecarboxamide;
1-[4-(methylamino)-6-phenyl-1,3,5-triazin-2-yl]-N-({2-[(trifluoromethyl)oxy]phenyl}methyl)-1,2,3,4-tetrahydro-7-quinolinecarboxamide;
1-[4-(4-chlorophenyl)-6-(methylamino)-1,3,5-triazin-2-yl]-N-[(2,4-dichlorophenyl)methyl]-1,2,3,4-tetrahydro-6-quinolinecarboxamide;
N-[(2,4-dichlorophenyl)methyl]-1-[4-(2-fluorophenyl)-6-(methylamino)-1,3,5-triazin-2-yl]-1,2,3,4-tetrahydro-6-quinolinecarboxamide;
N-[(2,4-dichlorophenyl)methyl]-1-{4-(methylamino)-6-[4-(methyloxy)phenyl]-1,3,5-triazin-2-yl}-1,2,3,4-tetrahydro-6-quinolinecarboxamide;
N-[(2,4-dichlorophenyl)methyl]-1-[4-(methylamino)-6-(4-pyridinyl)-1,3,5-triazin-2-yl]-1,2,3,4-tetrahydro-6-quinolinecarboxamide;
N-[(2,4-dichlorophenyl)methyl]-1-[4-(2,4-difluorophenyl)-6-(methylamino)-1,3,5-triazin-2-yl]-1,2,3,4-tetrahydro-6-quinolinecarboxamide;
2-(4-amino-6-methyl-1,3,5-triazin-2-yl)-N-({2-[(trifluoromethyl)oxy]phenyl}methyl)-2,3-dihydro-1H-isoindole-5-carboxamide;
2-(4-amino-6-methyl-1,3,5-triazin-2-yl)-N-({4-(1-pyrrolidinyl)-2-[(trifluoromethyl)oxy]phenyl}methyl)-2,3-dihydro-1H-isoindole-5-carboxamide;
2-(4-amino-6-methyl-1,3,5-triazin-2-yl)-N-{[2-chloro-4-(dimethylamino)phenyl]methyl}-2,3-dihydro-1H-isoindole-5-carboxamide;
2-(4-amino-6-methyl-1,3,5-triazin-2-yl)-N-{[4-chloro-2-(trifluoromethyl)phenyl]methyl}-2,3-dihydro-1H-isoindole-5-carboxamide;
N-({2-chloro-4-[(methylsulfonyl)amino]phenyl}methyl)-1-[4-methyl-6-(methylamino)-1,3,5-triazin-2-yl]-2,3-dihydro-1H-indole-5-carboxamide; and
1-(4-amino-6-methyl-1,3,5-triazin-2-yl)-N-({2-[(trifluoromethyl)oxy]phenyl}methyl)-2,3-dihydro-1H-indole-5-carboxamide;
or a pharmaceutically acceptable salt thereof.

5. A pharmaceutical composition comprising a compound or salt according to claim 1 and one or more pharmaceutically-acceptable excipient.

6. A method for treating hypertension, heart failure, peripheral vascular disease, coronary artery disease, myocardial ischemia, and angina comprising administering a safe and effective amount of a compound or salt according to claim 1 to a human in need thereof.

7. (canceled)

8. A method for treating renal or liver failure comprising administering a safe and effective amount of a compound or salt according to claim 1 to a human in need thereof.

9.-13. (canceled)

14. A method for preventing myocardial infarction or stroke comprising administering a safe and effective amount of a compound or salt according to claim 1 to a human in need thereof.

15. (canceled)

16. A method for treating COPD and asthma comprising administering a safe and effective amount of a compound or salt according to claim 1 to a human in need thereof.

17. A method for treating glucose intolerance, insulin insensitivity, diabetes and obesity comprising administering a safe and effective amount of a compound or salt according to claim 1 to a human in need thereof.

18. A method according to claim 6 wherein the compound is administered orally.

19. A method according to claim 6 wherein the compound is administered intravenously.

20. A method according to claim 6 wherein the compound is administered by inhalation.

21. A method according to claim 16 wherein the compound is administered orally.

22. A method according to claim 16 wherein the compound is administered intravenously.

23. A method according to claim 16 wherein the compound is administered by inhalation.
